SEQUENCE LISTING (12 sequences)

SEQ ID NO: 1 - DNA, 20 bp, Artificial
  Primer
aggtggttgcaactggacaa

SEQ ID NO: 2 - DNA, 20 bp, Artificial
  Primer
tcaggaactgttcgcccttc

SEQ ID NO: 3 - DNA, 20 bp, Artificial
  Primer
ataagcaccggctaactccg

SEQ ID NO: 4 - DNA, 20 bp, Artificial
  Primer
ttccatctgcctctccctca

SEQ ID NO: 5 - DNA, 19 bp, Artificial
  Primer
ggatgacacttttcggagc

SEQ ID NO: 6 - DNA, 18 bp, Artificial
  Primer
cattgtagcacgtgtgtc

SEQ ID NO: 7 - DNA, 24 bp, Artificial
  Primer
caaataaagttagaggtagaatgt

SEQ ID NO: 8 - DNA, 22 bp, Artificial
  Primer
ccataagcactagctagctgat

SEQ ID NO: 9 - DNA, 22 bp, Artificial
  Primer
gcaaaatccacagcttcatcgt

SEQ ID NO: 10 - DNA, 20 bp, Artificial
  Primer
ggaaggggtggtcatggaag

SEQ ID NO: 11 - DNA, 22 bp, Artificial
  Primer
ggtatgatttctacaaagcgag

SEQ ID NO: 12 - DNA, 21 bp, Artificial
  Primer
ataaaagactatcgtcgcgtg

(19) United States
(12) Patent Application Publication
(10) Pub. No.: US 2022/0184155 A1
(43) Pub. Date: Jun. 16, 2022

(54) BACTERIOPHAGES FOR INHIBITING BACTERIA

(71) Applicants: REGENTS OF THE UNIVERSITY OF MINNESOTA, Minneapolis, MN (US); SEOUL NATIONAL UNIVERSITY R&DB FOUNDATION, Seoul (KR)

(72) Inventors: Byeonghwa Jeon, St. Paul, MN (US); Sangryeol Ryu, Seoul (KR); Jinshil Kim, Seoul (KR)

(21) Appl. No.: 17/530,980

(22) Filed: Nov. 19, 2021

Related U.S. Application Data
(60) Provisional application No. 63/115,844, filed on Nov. 19, 2020.
(60) Provisional application No. 63/116,543, filed on Nov. 20, 2020.

(51) Int. Cl. A61K 35/76; C12N 1/20; A61P 31/04

(52) U.S. Cl. CPC A61K 35/76; A61P 31/04; C12N 1/20

(57) ABSTRACT

This disclosure describes bacteriophages that may be used to improve the selective isolation of Campylobacter from foods or to control antibiotic-resistant bacteria, or both. This disclosure further describes methods of using those bacteriophages.

### CROSS-REFERENCE TO RELATED APPLICATION

This application claims the benefit of U.S. Provisional Patent Application No. 63/115,844, filed Nov. 19, 2020, and U.S. Provisional Patent Application No. 63/116,543, filed Nov. 20, 2020, each of which is incorporated herein by reference in its entirety.

### BACKGROUND

Campylobacter is the primary cause of foodborne illnesses in the US and many other countries. Since Campylobacter is most frequently transmitted to humans by the consumption of contaminated foods, particularly poultry, Campylobacter detection may be used as a preventive measure. Campylobacter detection typically requires isolating and culturing Campylobacter, a fastidious bacterium that requires multiple growth conditions. Current selective media used for the isolating Campylobacter rely on the use of antimicrobials to which Campylobacter is intrinsically resistant. During growth of Campylobacter in culture, non-Campylobacter species of bacteria in, for example, a food sample also may be resistant to the antimicrobials in the culture medium and thus, may outgrow Campylobacter, reducing the isolation frequency of Campylobacter. Antibiotic-resistant E. coli, especially extended spectrum beta-lactamase (ESBL)-producing E. coli, is just one example of an antibiotic-resistant competitor that hampers selective isolation of Campylobacter from foods.

### SUMMARY

This disclosure describes bacteriophages that may be used to improve the selective isolation of Campylobacter from foods, to control antibiotic-resistant bacteria, or both. This disclosure further describes methods of using those bacteriophages.

In one aspect, this disclosure describes a composition that includes JEP1 (deposited in the NCBI database under GENBANK accession number MT740314), JEP4 (deposited in the NCBI database under GENBANK accession number MT740315), JEP6 (deposited in the NCBI database under GENBANK accession number MT764206), JEP7 (deposited in the NCBI database under GENBANK accession number MT764207), or JEP8 (deposited in the NCBI database under GENBANK accession number MT764208), or a combination thereof.

In some embodiments, the composition includes two, three, four, or all five of the phages. In some embodiments, the composition includes a divalent cationic ion. Exemplary divalent cationic ions include Ca²⁺, Mg²⁺, Cu²⁺, or Mn²⁺, or a combination thereof. In some embodiments, the composition includes CaCl₂) in a range of 0.1 mM to 15 mM or in a range of 0.1 mM to 10 mM. In some embodiments, the composition includes a Campylobacter-selective enrichment media.

In another aspect, this disclosure describes methods of using the composition.

In some embodiments, the method includes adding the composition to a Campylobacter-selective enrichment media. In some embodiments, a sample may also be added to the Campylobacter-selective enrichment media. In some embodiments, the sample may include an isolate from a food source. In some embodiments, the Campylobacter-selective enrichment media may be incubated at a temperature of at least 30° C., at least 33° C., at least 35° C., at least 37° C., at least 40° C., or at least 42° C., and up to 33° C., up to 35° C., up to 37° C., up to 40° C., up to 42° C., up to 45° C., or up to 47° C.; and/or incubated at a temperature of up to 33° C., up to 35° C., up to 37° C., up to 40° C., up to 42° C., up to 45° C., or up to 47° C. In some embodiments, the method may include adding an antibiotic to the Campylobacter-selective enrichment media.

In some embodiments, the method includes administering the composition to a subject. In some embodiments, the composition is administered in an amount effect to control the growth of extended-spectrum beta lactamase (ESBL)-producing E. coli. In some embodiments, the subject is a human, a companion animal, or a domesticated animal. In some embodiments, the composition is administered to a subject suffering from an ESBL-producing E. coli infection.

In another aspect, this disclosure describes adding JEP1 (deposited in the NCBI database under GENBANK accession number MT740314), JEP4 (deposited in the NCBI database under GENBANK accession number MT740315), JEP6 (deposited in the NCBI database under GENBANK accession number MT764206), JEP7 (deposited in the NCBI database under GENBANK accession number MT764207), or JEP8 (deposited in the NCBI database under GENBANK accession number MT764208), or a combination thereof to a composition.

In some embodiments, two, three, four, or all five of the phages may be added to the composition.

In some embodiments, the composition may include a food source. Exemplary food sources include raw meat, an aquatic product, a raw vegetable, a retail-level ready-to-eat food, a frozen food, and/or a mushroom.

In some embodiments, after adding JEP1, JEP4, JEP6, JEP7, or JEP8, or a combination thereof to the composition, the method includes storing the composition at a temperature of up to 4° C., up to 7° C., up to 10° C., or up to 42° C.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Host range of E. coli phages JEP1, JEP4, JEP6, JEP7, and JEP8. The phage infection of 67 strains of AMR E. coli isolated from retail chicken. The antimicrobial resistance patterns of the E. coli strains and the infectivity of five phages are indicated. CIP, ciprofloxacin; KAN, kanamycin; GEN, gentamicin; CHL, chloramphenicol; STR, streptomycin; TET, tetracycline; CTX, cefotaxime; AMP, ampicillin; and phylogroup U, Unknown.

FIG. 2. Host range of E. coli phages JEP1, JEP4, JEP6, JEP7, and JEP8. Association of the phylogenetic groups of AMR E. coli with the infection frequency of the five phages. The experiment was repeated three times. Statistical analysis was performed using the chi-square test with GraphPad Prism (*P<0.05; **P<0.01; ***P<0.001; ns, not significant).

FIG. 3. Morphological features of E. coli phages JEP1, JEP4, JEP6, JEP7, and JEP8. Transmission electron microscopy (TEM) images of each phage.

FIG. 4 shows phylogenetic association of E. coli phages JEP1, JEP4, JEP6, JEP7, and JEP8. VICTOR analysis was performed to analyze the phylogenetic association. All pairwise comparisons of the translated amino acid sequences of phages were conducted using the GBDP method (Meier-Kolthoff et al. BMC bioinformatics 14, 60 (2013)) under the settings recommended for prokaryotic viruses (Meier-Kolthoff et al. Bioinformatics 33, 3396-3404 (2017)). The resulting intergenomic distances were used to infer a balanced minimum evolution tree with branch support via FASTME including SPR postprocessing (Lefort et al. Mol Biol Evol 32, 2798-2800 (2015)) for formulas D4. Branch support was inferred from 100 pseudo-bootstrap replicates each. Trees were rooted at the midpoint (Farris The American Naturalist 106, 645-668 (1972)) and visualized with FigTree (Rambaut (2006), available online at tree.bio.ed.ac.uk/software/figtree/). Taxon boundaries at the species, genus and family level were estimated with the OPTSIL program (Göker et al. PLoS One 4, e6319 (2009)), the recommended clustering thresholds (Meier-Kolthoff et al. Bioinformatics 33, 3396-3404 (2017)) and an F value (fraction of links required for cluster fusion) of 0.5 (Meier-Kolthoff et al. Stand Genomic Sci 9, 2 (2014)).

FIG. 5. Inhibition of ESBL-producing E. coli in combination with antibiotics. (A) Inhibition of ESBL-producing E. coli by the phage cocktail in Preston selective broth. (B) The inhibition of ESBL-producing E. coli by the phage cocktail in Bolton selective broth. For (A) and (B), mixed-culture of ESBL-producing E. coli isolates was inoculated in each enrichment broth with SM buffer (control, gray bar) and the phage cocktail (10⁷ plaque forming units (PFU)/mL, black bar) at 42° C. for 24 hours under microaerobic conditions. The column and error bars represent the mean and the standard errors of the mean of triplicate experiments. Student's t-test was performed using GraphPad Prism in comparison with the non-treated control. *, P<0.05; **, P<0.01; ***, P<0.001; ND, not detected. (C) Checkerboard titration assay of the effect of the combination of phage and various antibiotics (rifampicin, cefoperazone, vancomycin, or polymyxin) on growth of ESBL E. coli mixed culture. The combination of phage and antibiotic was more effective than antibiotic alone in all cases with a high enough MOI.

FIG. 6. Inhibition of ESBL-producing E. coli by the phage cocktail in Campylobacter-selective enrichment broth. (A) The level of ESBL-producing E. coli was determined using qPCR amplifying uidA. (B) CFU counting on MacConkey agars supplemented with 1 μg/ml cefotaxime. SM buffer was added to the non-treated controls. Statistical significance was evaluated with Student's t-test using GraphPad Prism. *, P<0.05; **, P<0.01; ***, P<0.001; ns, not significant.

FIG. 7 shows the experimental levels of Campylobacter in different enrichment conditions. The level of Campylobacter spp. was determined with qPCR using 16s rRNA. SM buffer was added to negative controls without the phage cocktail. Student's t-test was performed using GraphPad Prism. ns, not significant.

FIG. 8 shows exemplary effects of phage cocktail treatment on Campylobacter isolation. Frequency of Campylobacter isolation from ten retail chicken samples by using Bolton and Preston media in the presence or absence of the phage cocktail under different enrichment conditions. SM buffer was added to samples for negative controls. Statistical analysis was performed using GraphPad Prism, and significance was tested by chi-square test by comparing the phage cocktail treated samples and non-treated samples. *, P<0.05. NI, not isolated.

FIG. 9. Bolton agar inoculated with a dilute of Preston selective enrichment broth without the phage cocktail. E. coli forms white colonies, whereas Campylobacter forms transparent colonies.

FIG. 10. Bolton agar inoculated with a dilute of Preston selective enrichment broth with the phage cocktail. E. coli forms white colonies, whereas Campylobacter forms transparent colonies.

FIG. 11. Phage map of JEP1.

FIG. 12. Phage map of JEP4.

FIG. 13. Phage map of JEP6.

FIG. 14. Phage map of JEP7.

FIG. 15. Phage map of JEP8.

FIG. 16. The legend for the phage maps in FIGS. 11-15.

FIG. 17. Effect of phage cocktail on viability of a mixed culture of AMR E. coli isolates (E20, E41, E55, and E59) in LB broth with (Phage cocktail) and without (control) the phage cocktail. (A) 25° C. (B) 4° C. Student's t-test was performed using GraphPad Prism in comparison with the non-treated control. *, P<0.05; **, P<0.01; ***, P<0.001.

FIG. 18. Effect of phage cocktail on viability of a mixed culture of AMR E. coli isolates (E20, E41, E55, and E59) in raw chicken skin with (Phage cocktail) and without (control) the phage cocktail. (A) 25° C. (B) 4° C. Student's t-test was performed using GraphPad Prism in comparison with the non-treated control. *, P<0.05; ***, P<0.001.

FIG. 19. Effect of divalent cationic ions on phage efficacy. Effect of 0.1 mM of CaCl₂) and MgCl₂ on the inhibition by the phage cocktail of a mixed culture of ESBL-producing E. coli (E20, E41, E55, and E59).

FIG. 20. Effect of varying concentrations of CaCl₂) on inhibition by the phage cocktail of a mixed culture of ESBL-producing E. coli (E20, E41, E55, and E59).

FIG. 21. Phage inhibition of mixed cultures of AMR E. coli strains in LB broth at 37° C. (A) Mixed culture 1 consisting of AMR E. coli strains E20, E41, E55, and E59. (B) Mixed culture 2 consisting of AMR E. coli strains E3, E43, E55, and E59. The reduction in the OD₆₀₀ of the mixed culture of AMR E. coli strains was measured after treatment with single phages or the phage cocktail, as shown. The data present the means and the standard errors of the mean (SEM) of the results of three experiments. Statistical analysis was performed using a Student's t-test compared to the control in the same sampling (12 h) with GraphPad Prism (***, P<0.001).

FIG. 22. Phage inhibition of mixed cultures of AMR E. coli strains in LB broth at 37° C. (A) Mixed culture 3 consisting of AMR E. coli strains E17, E41, E52, and E59. (B) Mixed culture 4 consisting of AMR E. coli strains E20, E45, E52, and E59. The reduction in the OD₆₀₀ of the mixed culture of AMR E. coli strains was measured after treatment with single phages or the phage cocktail. The data present the means and the standard errors of the mean (SEM) of the results of three experiments. Statistical analysis was performed using a Student's t-test compared to the control in the same sampling (12 h) with GraphPad Prism (**, P<0.01).

### DETAILED DESCRIPTION OF ILLUSTRATIVE EMBODIMENTS

This disclosure describes bacteriophages that may be used to improve the selective isolation of Campylobacter from foods or to control antibiotic-resistant bacteria, or both. This disclosure further describes methods of using those bacteriophages.

### Campylobacter Spp., Human Infection, and Culture

Campylobacter spp. are the leading bacterial pathogen causing gastroenteritis in humans and are annually responsible for 166 million infection cases, approximately one tenth of the total global diarrheal cases. Human infection with Campylobacter is characterized by severe abdominal cramping, watery or bloody diarrhea, and, in some cases, Guillain-Barré syndrome occurs as a post-infection complication. Among several pathogenic species of Campylobacter, Campylobacter jejuni is most frequently implicated in human infection. Because C. jejuni colonizes the poultry intestines as a non-pathogenic commensal bacterium, human exposure to C. jejuni is mainly associated with the ingestion of undercooked, contaminated poultry meat. A reduction in Campylobacter counts on chicken carcasses by two orders of magnitudes could decrease human infections with Campylobacter by 30-fold. Unlike other most foodborne pathogens, C. jejuni is considered fastidious due to its unique growth requirements. As a microaerophile, C. jejuni requires low oxygen levels but is sensitive to atmospheric oxygen tension, whereas facultative anaerobic pathogens, such as pathogenic Escherichia coli and Salmonella, can grow both aerobically and anaerobically. Moreover, C. jejuni does not use glucose as a nutrient and optimally grows at an elevated temperature. Overall, the fastidiousness of Campylobacter creates challenges in isolating Campylobacter from various food, environmental, and clinical samples.

Selective enrichment media are routinely used to isolate Campylobacter by generating selective pressure with antibiotics. For instance, Bolton Campylobacter-selective supplement contains cefoperazone, vancomycin, and trimethoprim, antibiotics to which Campylobacter is intrinsically resistant. However, the increasing prevalence of antibiotic-resistant bacteria markedly hinders the isolation of Campylobacter because Campylobacter is competitively outgrown by robust, faster growing bacteria. For example, extended-spectrum beta-lactamase (ESBL)-producing E. coli, which often contaminates poultry carcasses, is resistant to the third-generation cephalosporin drugs that supplement Campylobacter-selective media. Microbiota analysis demonstrates that the increased level of antibiotic-resistant E. coli during selective enrichment is an impediment to Campylobacter isolation, suggesting that suppression of the growth of ESBL-producing E. coli could enhance selective enrichment.

A typical method to isolate Campylobacter from food includes an enrichment step using either Bolton selective media or Preston selective media and a cultivation step with modified charcoal-cefoperazone-deoycholate agar (mCCDA). mCCDA includes cefoferazone, the same third-generation cephalosporin antibiotic used in Bolton media.

Exemplary methods of isolating Campylobacter are also described in ISO 10272-2:2017, entitled “Microbiology of the food chain—Horizontal method for detection and enumeration of Campylobacter spp.—Part 2: Colony-count technique” and in Chapter 7 of the FDA's Bacteriological Analytical Manual (BAM), available online at www.fda.gov/food/laboratory-methods-food/bam-chapter-7-campylobacter.

E. coli and Human Infection

In addition to Campylobacter spp., antibiotic-resistant bacteria in animals used as a food source may contaminate foods, resulting in humans being exposed to antibiotic-resistant bacteria when they consume contaminated food. Antibiotic-resistant foodborne pathogens may cause clinical problems because the resistance often limits the clinical options for antimicrobial chemotherapy of serious cases, Moreover, non-pathogenic, antibiotic-resistant enteric bacteria on foods are a reservoir for antibiotic resistance and spread antibiotic resistance genes.

Antibiotic resistance genes encoded on mobile genetic elements, such as plasmids, are easily transmitted among bacterial populations by horizontal gene transfer. For instance, extended spectrum beta-lactamase (ESBL), which is usually encoded on plasmids, confers resistance to all beta-lactam drugs except carbapenems, and ESBL-producing Escherichia coli is highly prevalent in poultry. To stop the spread of antibiotic resistance, it is desired to have additional means of controlling antibiotic-resistant bacteria in the food supply chain.

### Bacteriophages

In one aspect, this disclosure describes bacteriophages that may be used to improve the selective isolation of Campylobacter from foods or to control antibiotic-resistant bacteria, or both. These bacteriophages include five Myoviridae phages: JEP1, JEP4, JEP6, JEP7, and JEP8. In some embodiments, the bacteriophages are included in a single composition, also referred to herein as a “phage cocktail,” as further described herein.

Bacteriophages (also referred to as phages) are viruses that specifically infect target bacteria. Phages infect host bacteria depending on the availability of specific phage receptors on the bacterial surface. Whereas antibiotics, particularly those with a broad spectrum, unnecessarily eliminate beneficial commensal bacteria along with target pathogens, the host specificity of phage therapy can inhibit only target bacteria without disrupting normal microbiota. On the other hand, the host range of phage infection is frequently too narrow to cope with antibiotic-resistant bacteria with a wide genetic diversity. To widen the spectrum of phage therapy, the most common approach is to construct cocktails using phages with different host ranges.

Example 1 describes screening the phage infectivity of 67 strains of ESBL-producing E. coli, which revealed that E. coli phages exhibit differences in infection efficiency depending on the bacterial phylogenetic group. Example 1 further describes the development of a phage cocktail that includes JEP1, JEP4, JEP6, JEP7, and JEP8, phages that primarily infect the major phylogroups of ESBL-producing E. coli from poultry.

As further described in Example 1, the efficacy of Campylobacter isolation is increased in the presence of the phage cocktail. As further described in Example 2, the growth of extended-spectrum beta lactamase (ESBL)-producing (i.e., antibiotic-resistant) E. coli is inhibited in the presence of the phage cocktail.

### Composition Including the Bacteriophages (“Phage Cocktail”)

In another aspect, this disclosure describes a composition that includes JEP1, JEP4, JEP6, JEP7, or JEP8, or a combination thereof. Compositions including a combination of the phages, also referred to herein as a “phage cocktail,” may include two, three, four, or all five of the phages. In some embodiments, a composition including a phage cocktail including all five of the phages (JEP1, JEP4, JEP6, JEP7, and JEP8) may be preferred.

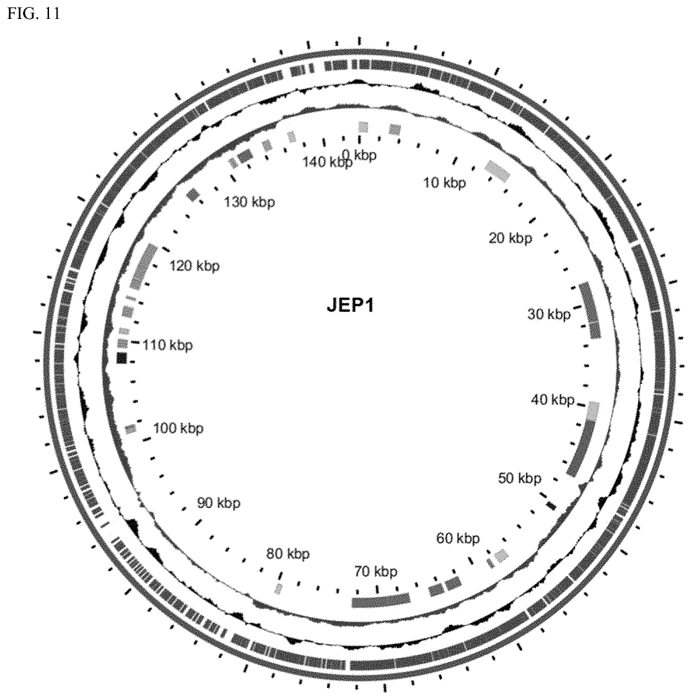

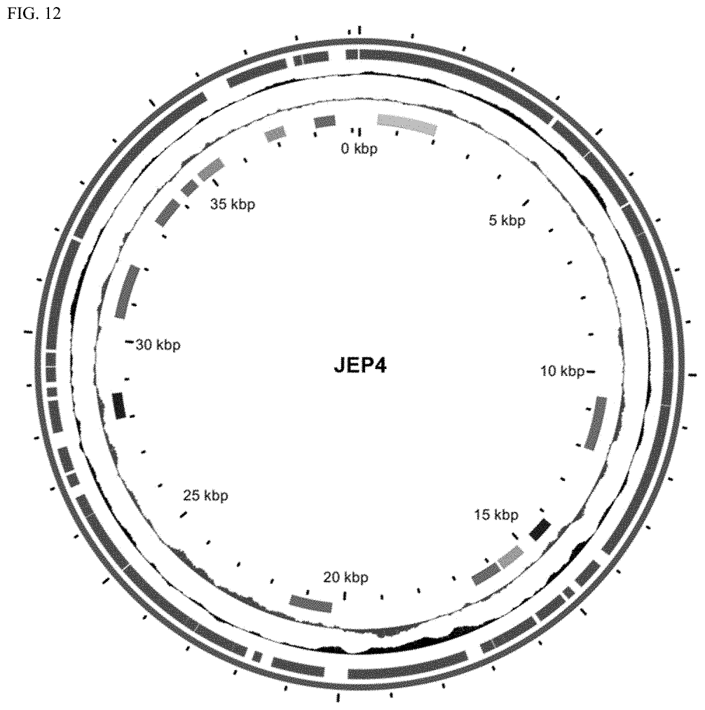

JEP1 is deposited in the NCBI database under GENBANK accession number MT740314, a phage map of which is shown in FIG. 11.

JEP4 is deposited in the NCBI database under GENBANK accession number MT740315, a phage map of which is shown in FIG. 12.

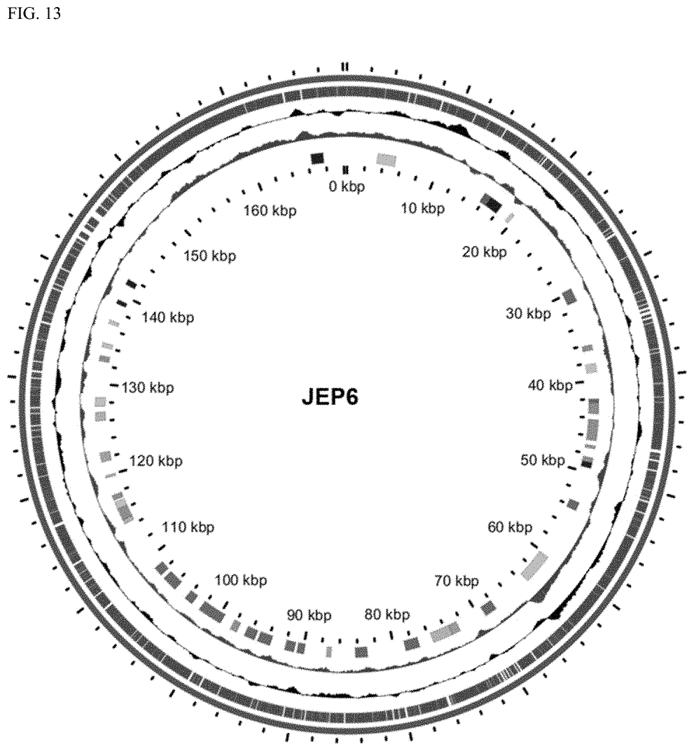

JEP6 is deposited in the NCBI database under GENBANK accession number MT764206, a phage map of which is shown in FIG. 13.

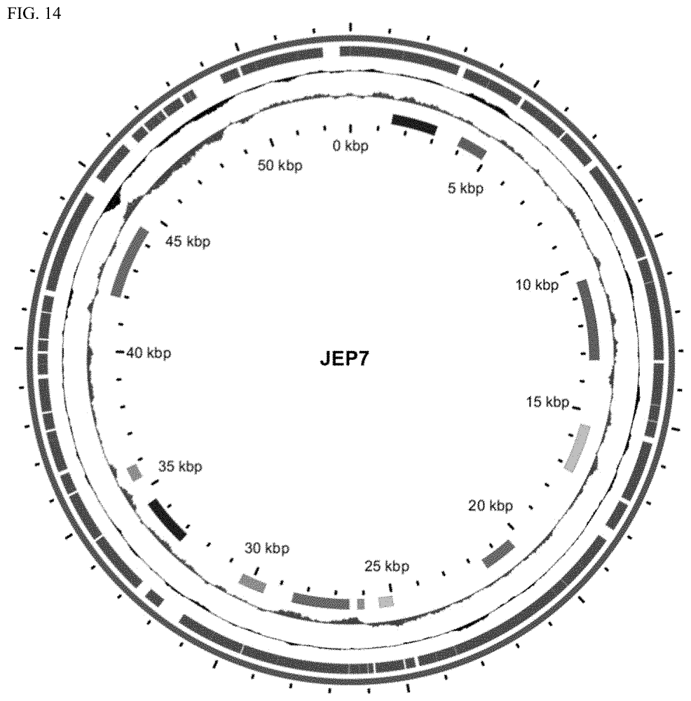

JEP7 is deposited in the NCBI database under GENBANK accession number MT764207, a phage map of which is shown in FIG. 14.

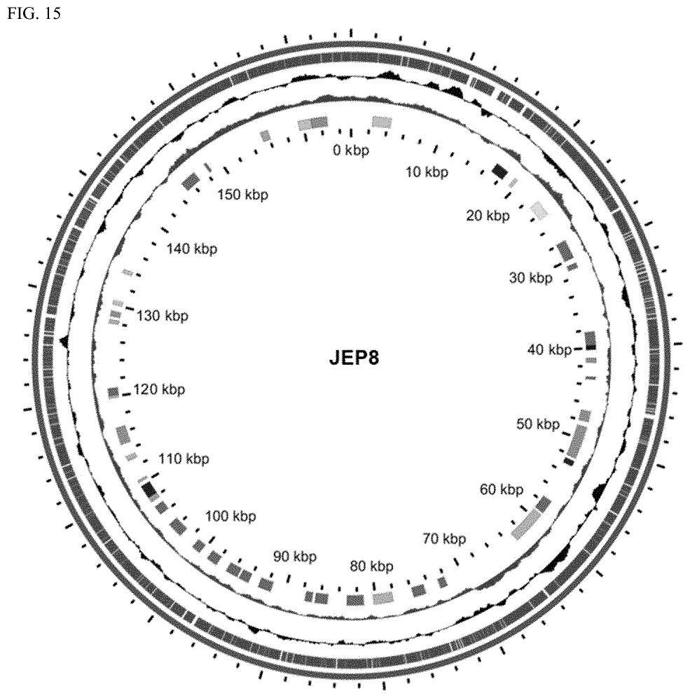

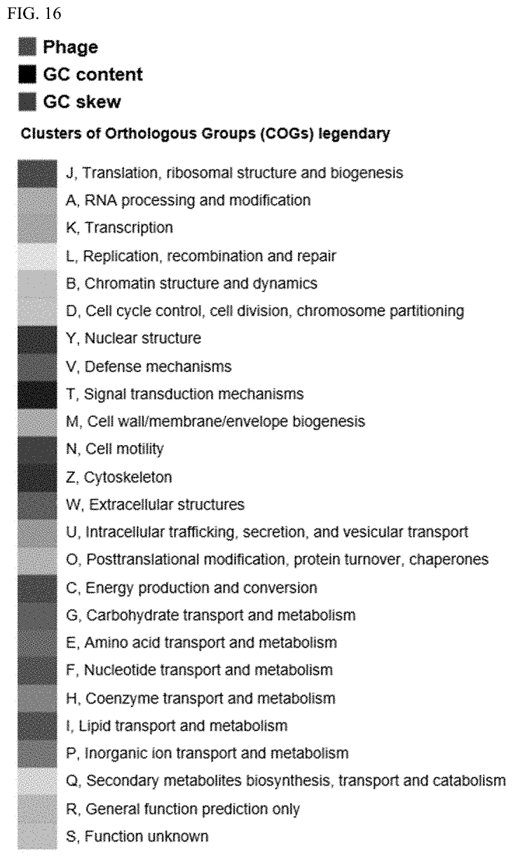

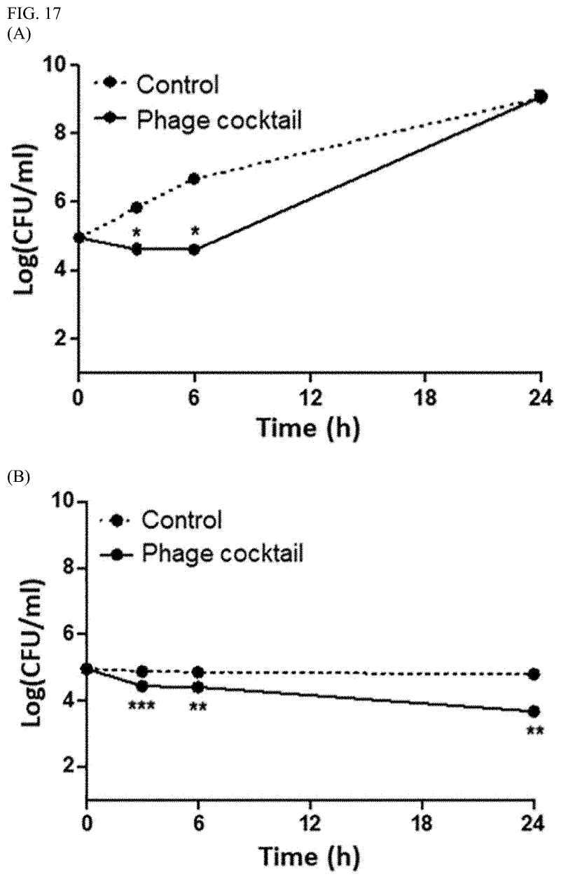

JEP8 is deposited in the NCBI database under GENBANK accession number MT764208, a phage map of which is shown in FIG. 15.

In some embodiments, the composition may include a divalent cationic ion. Exemplary divalent cationic ions include Ca²⁺, Mg²⁺, Cu²⁺, Mn²⁺, or a combination thereof. In certain embodiments, the divalent cationic ion includes Ca²⁺.

The concentration of the divalent cationic ion (or of, for example, CaCl₂), MgCl₂, CuCl₂, or MnCl₂ in the solution) may be selected depending on the intended use of the composition. In some embodiments, the composition may include at least 0.01 mM CaCl₂), at least 0.1 mM CaCl₂), or at least 1 mM CaCl₂). In some embodiments, the composition may include up to 0.1 mM CaCl₂), up to 1 mM CaCl₂), up to 5 mM CaCl₂), up to 10 mM CaCl₂), or up to 15 mM CaCl₂). For example, the composition may include CaCl₂) in a range of 0.1 mM to 15 mM or in a range of 0.1 mM to 10 mM.

As shown in Example 3, when the phage cocktail included of 0.1 mM CaCl₂), the phage cocktail postponed the emergence of E. coli tolerant to phage infection until 20 hours (compared to 12 hours without CaCl₂)) and significantly intensified phage inhibition of the E. coli bacteria.

### Methods of Using the Bacteriophages or a Composition Including the Bacteriophages

In another aspect this disclosure describes methods of using the bacteriophages described herein (JEP1, JEP4, JEP6, JEP7, or JEP8, or a combination thereof) and of using a composition including those bacteriophages. In some aspects, one or more of the bacteriophages may be used to control the growth of extended-spectrum beta-lactamase (ESBL)-producing E. coli. The growth of ESBL-producing E. coli may be controlled in a composition such as a food source. Or an effective amount of a composition including one or more of the phages may be administered to a subject to control the growth of ESBL-producing E. coli.

### Selective Isolation

In some embodiments, JEP1, JEP4, JEP6, JEP7, JEP8, or a combination thereof may be used to control the growth of extended-spectrum beta-lactamase (ESBL)-producing E. coli during the selective isolation of Campylobacter from a sample.

Exemplary samples include an isolate from a food source, a clinical sample, or an environmental sample. Exemplary food sources include poultry carcasses. Exemplary clinical samples include fecal samples including, for example, from diarrheal subjects. Subject may include human or animals. Animals may include, for example, companion animals (such as dogs) or livestock. Exemplary environmental samples include ground samples, surface samples, and drinking water samples. In some embodiments, the sample is expected to include Campylobacter.

In some embodiments, JEP1, JEP4, JEP6, JEP7, JEP8, or a combination thereof may be added to a Campylobacter-selective enrichment media. A Campylobacter-selective enrichment media may include a liquid media (or broth) or a solid media. Exemplary Campylobacter-selective enrichment broths include Preston selective broth and Bolton selective broth. Exemplary Campylobacter-selective solid media include Bolton agar (BA), Preston agar, and modified charcoal-cefoperazone-deoycholate agar (mCCDA). In some embodiments, the Campylobacter-selective enrichment media may preferably include Preston selective broth. As further described in Example 1, E. coli growth in Preston selective broth was significantly inhibited by the addition of phage cocktail when the broth was incubated at both 37° C. and 42° C.

JEP1, JEP4, JEP6, JEP7, JEP8, or a combination thereof may be added to a Campylobacter-selective enrichment media at any suitable concentration. For instance, the concentration may vary depending on the enrichment media used. In some embodiments, the Campylobacter-selective enrichment media may include at least 10⁴ plaque forming units (PFU)/mL. Exemplary concentrations of JEP1, JEP4, JEP6, JEP7, JEP8, or a combination thereof that may be used include up to 10⁷ plaque forming units (PFU)/mL, up to 10⁸ PFU/mL, up to 10⁹ PFU/mL, up to 10¹⁰ PFU/mL, or up to 10¹¹ PFU/mL of each bacteriophage included in the Campylobacter-selective enrichment media. For example, if the Campylobacter-selective enrichment media includes JEP1 and JEP4, the media might include up to 10⁸ PFU/mL of JEP1 and up to 10⁸ PFU/mL of JEP4. If the Campylobacter-selective enrichment media includes JEP1, JEP4, JEP6, JEP7, and JEP8, the Campylobacter-selective enrichment media might include up to 10⁸ PFU/mL of each of JEP1, JEP4, JEP6, JEP7, and JEP8. Because a higher PFU may increase the cost, in some embodiments, a concentration of up to 10⁸ PFU/mL of each bacteriophage may be preferred to balance cost with efficacy.

In some embodiments, after adding JEP1, JEP4, JEP6, JEP7, JEP8, or a combination thereof to a Campylobacter-selective enrichment media, the Campylobacter-selective enrichment media may be incubated at a temperature of at least 30° C., at least 33° C., at least 35° C., at least 37° C., at least 40° C., or at least 42° C. In some embodiments, after adding JEP1, JEP4, JEP6, JEP7, or JEP8, or a combination thereof to a Campylobacter-selective enrichment media, the Campylobacter-selective enrichment media may be incubated at a temperature of up to 33° C., up to 35° C., up to 37° C., up to 40° C., up to 42° C., up to 45° C., or up to 47° C. For example, the Campylobacter-selective enrichment media may be incubated at a temperature in a range of 35° C. to 45° C., in a range of 35° C. to 40° C., or in a range of 40° C. to 45° C. As further described in Example 1, the phage cocktail reduced the level of E. coli in Bolton selective broth incubated at 42° C. and in Preston selective broth incubated at both at 37° C. and 42° C.

In some embodiments, JEP1, JEP4, JEP6, JEP7, JEP8, or a combination thereof may be added in combination with an antibiotic. For example, JEP1, JEP4, JEP6, JEP7, JEP8, or a combination thereof and an antibiotic may be added to the same Campylobacter-selective enrichment media. Exemplary antibiotics that may be used include, for example, cefoperazone, vancomycin, polymyxin B, rifampicin, bacitracin, cephalothin, cephazolin, colistin, and novobiocin. As further described in Example 1, the phage cocktail may exhibit antimicrobial synergy in combination with an antibiotic. For example, synergistic inhibition was observed when the phage cocktail was used in combination with rifampicin in Preston selective broth.

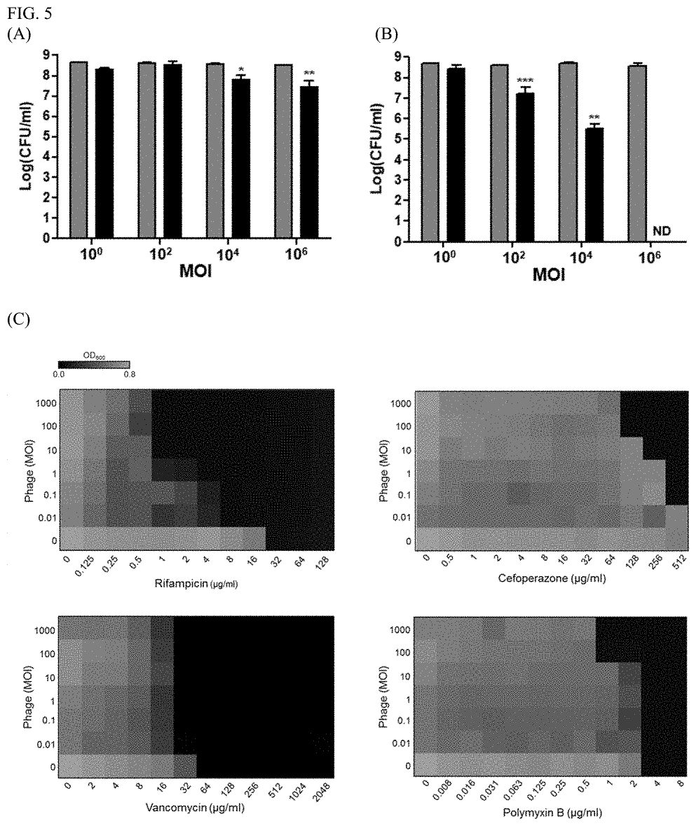

For example, FIG. 5C shows data relating to the effects of various combinations of phage and an antibiotic against a mixed culture of ESBL-E. coli. The antibiotics were selected based on the presence of the selected antibiotics as the selective supplements used in Bolton Campylobacter-selective broth and Preston Campylobacter-selective broth. The growth of a mixed culture of ESBL-E. coli (dark indicates no or reduced growth) is reduced with the combination of phage and antibiotics. The most substantial effect was observed when the phage cocktail was combined with rifampicin, an antibiotic supplement in Preston Broth (FIG. 5C). Although the minimum inhibitory concentration (MIC) of cefoperazone was high (>512 μg/ml) due to cephalosporin resistance conferred by ESBL, the susceptibility of ESBL-producing E. coli to cefoperazone was increased when the phages were used at high MOIs (FIG. 5C). The co-treatment with the phage cocktail reduced the MIC of vancomycin by two-fold (FIG. 5C) and the MIC by four-fold (FIG. 5C). These results indicate that phages can enhance the antimicrobial activity of antibiotics when used together. Thus, phages can be used complimentarily with antibiotics.

In some embodiments, the method further includes adding the sample to the Campylobacter-selective enrichment media. The Campylobacter-selective enrichment media may be prepared according to standard protocols, including, for example, as described in Chapter 7 of the FDA's Bacteriological Analytical Manual (BAM), available online at www.fda.gov/food/laboratory-methods-food/bam-chapter-7-campylobacter.

In some embodiments, a method of selectively isolating Campylobacter from a sample may include culturing the sample in a series of selective media. For example, the sample may be cultured in one or more Campylobacter-selective enrichment liquid media and/or cultured on one or more Campylobacter-selective enrichment solid media. JEP1, JEP4, JEP6, JEP7, JEP8, or a combination thereof may be added to one or more of these selective broths and/or media.

In one exemplary embodiment, a sample may be added to a liquid Campylobacter-selective enrichment media that includes JEP1, JEP4, JEP6, JEP7, JEP8, or a combination thereof. A portion of this liquid enrichment media may ben be added to a second Campylobacter-selective enrichment media. The second Campylobacter-selective enrichment media may preferably be a solid media.

In another exemplary method of selectively isolating Campylobacter from a culture, the phage cocktail including JEP1, JEP4, JEP6, JEP7, and JEP8 may be added with a sample to a Preston selective broth before the resulting culture is transferred to a Bolton agar (BA) selective solid media. While in the broth, the sample may be incubated at 42° C. As further described in Example 3, the highest frequency of Campylobacter isolation was observed when the samples were enriched at 42° C. in Preston selective broth supplemented with the phage cocktail and then subsequently cultured on BA.

### Control of Antibiotic-Resistant Bacteria in a Composition

In some embodiments, JEP1, JEP4, JEP6, JEP7, JEP8, or a combination thereof may be used to control the growth of extended-spectrum beta-lactamase (ESBL)-producing E. coli in a composition.

An exemplary composition includes a food source. The food source may be any food source susceptible to contamination with antibiotic-resistant bacteria and, in particular, ESBL-producing E. coli. Exemplary food sources include raw meat (for example, raw chicken), aquatic products, raw vegetables, retail-level ready-to-eat foods, frozen foods, and mushrooms. Combinations of these food sources can also be envisioned. Other food sources include animal feed, pet food, etc.

In some embodiments, the food source may be treated with a phage composition including JEP1, JEP4, JEP6, JEP7, JEP8, or a combination thereof.

The phage composition may include the phage or phages at any suitable concentration. The phage composition may include at least 10⁴ plaque forming units (PFU)/mL. Exemplary phage compositions may include up to 10⁷ PFU/mL, up to 10⁸ PFU/mL, up to 10⁹ PFU/mL, up to 10¹⁰ PFU/mL, or up to 10¹¹ PFU/mL of each phage included in the phage composition. For example, if the phage composition included JEP1 and JEP4, the phage composition might include up to 10⁸ PFU/mL of JEP1 and up to 10⁸ PFU/mL of JEP4. If the phage composition included JEP1, JEP4, JEP6, JEP7, and JEP8, the phage composition might include up to 10⁸ PFU/mL of each of JEP1, JEP4, JEP6, JEP7, and JEP8.

The phage composition may be a buffered solution. An exemplary buffered solution includes SM buffer (which includes a mixture of sodium chloride, magnesium sulphate and gelatin), Tris, HEPES, etc. In some embodiments, SM buffer may be preferred.

The phage composition may include a divalent cationic ion. Exemplary divalent cationic ions include Ca²⁺, Mg²⁺, Cu²⁺, Mn²⁺, or a combination thereof. In certain embodiments, the divalent cationic ion includes Ca²⁺.

The concentration of the divalent cationic ion (or of, for example, CaCl₂), MgCl₂, CuCl₂, or MnCl₂ in the solution) may be selected depending on the intended use of the composition. In some embodiments, the composition may include at least 0.01 mM CaCl₂), at least 0.1 mM CaCl₂), or at least 1 mM CaCl₂). In some embodiments, the composition may include up to 0.1 mM CaCl₂), up to 1 mM CaCl₂), up to 5 mM CaCl₂), up to 10 mM CaCl₂), or up to 15 mM CaCl₂). For example, the composition may include CaCl₂) in a range of 0.1 mM to 15 mM or in a range of 0.1 mM to 10 mM.

As shown in Example 3, CaCl₂) may be particularly effective at intensifying phage inhibition of E. coli bacteria.

In some embodiments, after JEP1, JEP4, JEP6, JEP7, JEP8, or a combination thereof, the food source may preferably be stored at a temperature of up to 4° C., up to 7° C., or up to 10° C. In some embodiments, the food source may preferably be stored at a temperature of at least 0° C.

As described in Example 2, the phage cocktail may be particularly useful in certain industrial settings where raw meat (for example, raw chicken) products are processed and/or distributed at refrigeration temperatures because the phage cocktail inhibited ESBL-producing E. coli more efficiently at 4° C. than 25° C. on chicken skin.

### Administration to a Subject and Therapeutic Uses

In some embodiments, a composition including JEP1, JEP4, JEP6, JEP7, JEP8, or a combination thereof may be administered to a subject. The composition may be used as a therapeutic to control the growth of extended-spectrum beta-lactamase (ESBL)-producing E. coli. For example, an effective amount of the composition including one or more of the phages may be administered to a subject to control the growth of ESBL-producing E. coli.

When ESBL-producing E. coli cause human infection, patients often require hospitalization, intravenous antibiotics such as carbapenem, and/or combinations of antibiotics. ESBL-producing E. coli are also frequently associated with healthcare-acquired infections (also known as nosocomial infections) which are among the most difficult problems confronting clinicians who deal with severely ill patients. Thus, additional methods to treat ESBL-producing E. coli infections would be beneficial.

### Administration

A composition including JEP1, JEP4, JEP6, JEP7, JEP8, or a combination thereof as an active agent may be administered to a subject. In some embodiments, the composition may include a pharmaceutically acceptable carrier.

The composition may be administered to any suitable subject including, for example, a vertebrate, more preferably a mammal, such as a human, a companion animal, a domesticated animal, and/or an animal in the wild in an amount effective to produce the desired effect.

Companion animals include, but are not limited to, dogs, cats, hamsters, gerbils, and guinea pigs. Domesticated animals include, but are not limited to, cattle, horses, pigs, goats, poultry (such as chicken, turkeys, geese, ducks, etc.), and llamas. Research animals include, but are not limited to, mice, rats, dogs, apes, and monkeys.

The composition may be administered in a variety of routes, including orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

The composition may can be administered as a single dose or in multiple doses. Useful dosages of the active agent may be determined by comparing their in vitro activity and the in vivo activity in animal models. Methods for extrapolation of effective dosages in mice, and other animals, to humans are known in the art.

Dosage levels of the active agent in a composition may be varied so as to obtain an amount of the active agent which is effective to achieve the desired therapeutic response for a particular subject, composition, and mode of administration, without being toxic to the subject. The selected dosage level will depend upon a variety of factors including the activity of the bacteriophage employed; the route of administration; the time of administration; the rate of excretion; the duration of the treatment; other drugs, compounds, and/or materials used in combination with the active agent; the age, sex, weight, condition, general health, and prior medical history of the subject being treated; and like factors well known in the medical arts.

### Methods of Treatment

JEP1, JEP4, JEP6, JEP7, JEP8, or a combination thereof may be used to treat or prevent infections with extended-spectrum beta-lactamase (ESBL)-producing E. coli. ESBL-producing E. coli infections include, but are not limited to, urinary tract infections, skin infections, pneumonias, gastrointestinal infections, and blood infections.

In some embodiments, a composition including JEP1, JEP4, JEP6, JEP7, JEP8, or a combination thereof may be used to treat a subject suffering from an infection with an ESBL-producing E. coli by administering the composition to the subject. Therapeutic treatment is initiated after diagnosis or the development of symptoms or clinical sign of infection.

Additionally or alternatively, a composition including JEP1, JEP4, JEP6, JEP7, JEP8, or a combination thereof may be administered prophylactically, delay, to reduce the likelihood, reduce the extent, or reduce the severity of infection. Treatment that is prophylactic, for instance, can be initiated before a subject manifests symptoms or clinical signs of infection. An example of a subject that is at particular risk is an immunocompromised person or a hospitalized person.

Treatment may be performed before, during, or after the diagnosis or development of symptoms of infection. Treatment initiated after the development of symptoms may result in decreasing the severity of the symptoms or clinical signs of infection, or completely resolving the symptoms or clinical signs of infection.

Administration of a composition including JEP1, JEP4, JEP6, JEP7, or JEP8, or a combination thereof may occur before, during, and/or after other treatments. Such combination therapy can involve the administration of an aurone during and/or after the use of other anti-bacterial agents. The administration the composition may be separated in time from the administration of other anti-bacterial agents by hours, days, or even weeks. Exemplary anti-bacterial agents that may be administered in combination with the composition include antibiotics.

The present invention is illustrated by the following examples. It is to be understood that the particular examples, materials, amounts, and procedures are to be interpreted broadly in accordance with the scope and spirit of the invention as set forth herein.

### EXAMPLES

All reagents, starting materials, and solvents used in the following examples were purchased from commercial suppliers (such as Sigma Aldrich, St. Louis, Mo.) and were used without further purification unless otherwise indicated.

### Example 1—Bacterial Phylogenetic Group-Based Construction of Bacteriophage Cocktails to Improve Campylobacter Isolation

This Example describes a method of Campylobacter isolation that includes suppressing competitive, antibiotic-resistant E. coli with a bacteriophage (phage) cocktail. As further described in this Example, to develop a phage cocktail capable of infecting various phylogenetic groups of extended-spectrum beta lactamase (ESBL)-producing E. coli, an extensive phage infection assay was performed with 67 ESBL-producing E. coli strains isolated from poultry, leading to selection of five Myoviridae phages (JEP1, JEP4, JEP6, JEP7, and JEP8). Each phage exhibited differential infection efficiency depending on the phylogenetic group of E. coli. JEP1 and JEP7 phages more effectively infected phylogroup B1 (69% and 77%, respectively) than other groups, while JEP4 showed a high infection frequency on phylogroup A (74%). The combination of phages efficiently infecting the major phylogenetic groups (e.g., A and B1) with those infecting the minor groups significantly improved the efficiency (91%) of phage inhibition of ESBL-producing E. coli. Moreover, the phage cocktail synergistically inhibited ESBL-producing E. coli in combination with antibiotics. The supplementation of phages in selective media markedly improved the frequency of Campylobacter isolation.

### Bacterial Strains and Growth Conditions

The ESBL-producing E. coli strains used in this work were isolated as described in Park et al. (Park et al. J Glob Antimicrob Resist 17, 216-220 (2019)). E. coli strains were routinely cultured at 37° C. in Luria-Bertani (LB) media (Difco, USA). ESBL-producing E. coli in enrichment cultures was quantified by serial dilution and plating on MacConkey agars supplemented 1 μg/mL cefotaxime. LB soft top agar plates were prepared with LB broth supplemented with 0.4% agar. C. jejuni was cultured at 42° C. in Mueller-Hinton (MH) media (Oxoid Limited, Basingstoke, UK) under microaerobic conditions (5% O₂, 10% CO₂, 85% N₂). All bacterial strains were stored at −80° C. in LB or MH broth with 15% glycerol.

### Phage Isolation

Phages were isolated from food (retail chicken and duck carcasses) and environment (chicken and pig feces) samples as described previously (Kim et al. Appl Environ Microbiol 77, 2042-2050 (2011)). Briefly, the samples were homogenized by vortexing in sodium chloride-magnesium sulfate (SM) buffer (100 mM NaCl, 10 mM MgSO₄.7H₂O, and 50 mM Tris.HCl, pH 7.5). After centrifugation at 10,000×g for 5 min, the supernatant was filtered through 0.22 μm pore size filters (MilliporeSigma, Burlington, Mass.). Five milliliters of filtered samples were mixed with equal volumes of 2×LB broth and 100 μl overnight culture of host. After incubation at 37° C. for overnight, the culture was centrifuged and filter-sterilized. To confirm the presence of phages, supernatants were serially diluted and spotted on 0.4% LB soft top agar containing overnight culture of ESBL-producing E. coli strains as hosts. After incubation at 37° C. for overnight, single plaque was picked and eluted with 1 ml SM buffer. The purification step was repeated at least three times for each phage.

To propagate phages, purified lysate was added to the culture of the host strains, and the mixture was incubated at 37° C. for four hours or overnight. Phage propagation was performed serially with three different culture volumes (4 mL, 40 mL, and 250 mL), and then the culture was centrifuged and filtered. Phage particles were precipitated by mixing with polyethylene glycol (PEG) 6000 (Junsei, Japan) and 1 M NaCl. Finally, CsCl density gradient ultracentrifugation (Himac CP 100b, Hitachi, Japan) with CsCl step densities (1.3 g/mL, 1.45 g/mL, 1.5 g/mL, and 1.7 g/mL) at 78,500×g was conducted at 4° C. for two hours. A band of viral particles was collected and dialyzed twice for one hour in 1 L of SM buffer. Concentrated phage stocks were stored at 4° C.

### Phage Host Range

A total of 67 strains of ESBL-producing E. coli were used to assess the host range. Each strain was incubated at 37° C. overnight with shaking, and then 100 μL of each bacterial culture was added to 5 mL of 0.4% LB soft agar and mixed. The mixture was overlaid on a 1.5% LB agar plate and dried at room temperature for 20 minutes. Subsequently, serially 10-fold diluted phage lysates were spotted onto the prepared bacterial lawn and incubated at 37° C. for 12 hours. After incubation, the formation of single plaques was recorded to determine phage sensitivity of each strain. The efficiency of phage infectivity (EOP) of each strain was compared to that of the host strains.

### Transmission Electron Microscopy (TEM)

The CsCl-purified phages were morphologically characterized by TEM analysis. A high titer of phage stock (about 1×10¹⁰ plaque forming unit (PFU)/mL) was placed on carbon-coated copper grids (200 mesh) and negatively stained with 2% aqueous uranyl acetate (pH 4.0) for 10 seconds. The phages were observed with energy-filtering TEM (LIBRA 120, Carl Zeiss, Germany) at 120-kv accelerating voltage at National Instrumentation Center for Environmental Management (Seoul, South Korea). The phages were identified and classified using the International Committee on Taxonomy of Viruses (ICTV) classification (King et al. (eds.), Virus taxonomy: Ninth report of the International Committee on Taxonomy of Viruses (2011)).

### Phage Cocktail

Equal amounts of CsCl-purified phages JEP1 (deposited in the NCBI database under GENBANK accession number MT740314), JEP4 (deposited in the NCBI database under GENBANK accession number MT740315), JEP6 (deposited in the NCBI database under GENBANK accession number MT764206), JEP7 (deposited in the NCBI database under GENBANK accession number MT764207), and JEP8 (deposited in the NCBI database under GENBANK accession number MT764208) were combined to form a 10⁸ PFU/mL composition in SM buffer.

### Inhibition Assay in Bolton and Preston Campylobacter-Selective Media

After the cultivation of the mixed culture of E. coli strains (E20, 41, 55, and 59), which were selected from major phylogroups A, B1, B2, and D to an optical density at 600 nm (OD₆₀₀) of 0.5, the mix-culture was diluted and added to 3 mL of Bolton Campylobacter-selective supplements (Oxoid Limited, Basingstoke, UK) and Preston broth with Preston Campylobacter-selective supplements (Thermo-Fisher Scientific, Waltham, Mass.) at 10¹, 10³, 10⁵, and 10⁷ colony forming units (CFU)/mL, followed by the addition of the phage cocktail at 10⁷ PFU/mL. After incubation at 42° C. for 24 hours, a 10-fold serial diluted culture was plated onto LB agar plate to enumerate the CFU. Approximately 10 grams of retail raw chicken was enrichment with 90 mL Bolton and Preston broth with phage cocktail or SM buffer at different temperature (37° C. or 42° C.) for 24 hours under microaerobic condition.

### Checkerboard Titration Assay

A checkerboard titration assay was performed as described previously (Hsieh et al. Diagn Microbiol Infect Dis 16, 343-349 (1993)). Briefly, antibiotics (cefoperazone, vancomycin, polymyxin B, and rifampicin) were two-fold serially diluted on each column, and phage cocktail was 10-fold diluted on each row. The mix-culture of E. coli strains (E20, 41, 55, and 59) was added to 10⁵ CFU/well, and the plate was incubated at 42° C. for 24 hours. The OD₆₀₀ was measured with SpectaMax i3 Platfrom (Molecular Devices, San Jose, Calif.), and shown as a heat plot.

Quantitative PCR (qPCR) to Determine the Levels of E. coli and Campylobacter in Enrichment Broth

qPCR was performed as described previously (Taskin et al. Appl Environ Microbiol 77, 4329-4335 (2011)). The standards for DNA copy number for qPCR were prepared using the E. coli K-12 W3110 or C. jejuni NCTC 11168 as control strain. To enumerate CFU, E. coli strain was grown to an OD₆₀₀ of 1, and overnight cultures of C. jejuni stains were harvested from MH agar plates and resuspended in MH broth to an OD₆₀₀ of 0.1. gDNA was extracted using the G-Spin™ Genomic DNA Extraction Kit (Intronbio, South Korea) according to the manufacturer's instructions. The purified gDNA was serially diluted to create a standard curve. gDNA copy number and cell number standard curves were generated with uidA (E. coli) and 16s rRNA (Campylobacter spp.) (Taskin et al. Appl Environ Microbiol 77, 4329-4335 (2011)). The qPCR reaction mixture contained 10 μL 2×iQ SYBR Green Supermix (Bio-Rad, Hercules, Calif.) and 0.3 μM of each primer in a reaction volume of 20 μL; uidA-qPCR-F and uidA-qPCR-R or 16s-qPCR-F and 16s-qPCR-R (Table 1). All qPCRs were performed using the CFX Connect™ Real-Time PCR detection system (Bio-Rad, Hercules, Calif.), and the cycling parameters were as follows: 95° C. for five minutes; 39 cycles at 95° C. for 15 seconds, 55° C. for 15 seconds, 72° C. for 30 seconds; 72° C. for seven minutes. The enrichment cultures (20 mL) were concentrated by centrifugation at 4,000×g, 4° C. for seven minutes, and pellets were resuspended with 2 mL of PBS. gDNA was extracted from 1 ml resuspension and analyzed by qPCR.

### Isolation of Campylobacter Under 16 Different Enrichment Combinations

Sixteen different enrichment conditions were examined to compare the isolation frequency by combining the first enrichment media (Bolton broth or Preston broth), at 37° C. or 42° C., with or without the phage cocktail, followed by the cultivation on the second enrichment media (Bolton agar or Preston agar) at 42° C. An aliquot (20 mL) of enrichment media was concentrated and resuspended in 2 mL of PBS. After 10-fold serial dilution with PBS, bacterial suspension (100 μL) was spread onto Bolton and Preston agars supplemented with each Campylobacter-selective supplement. The culture was incubated at 42° C. for 48 hours under microaerobic conditions. Based on the colony morphology, 15 presumptive C. jejuni colonies were confirmed by multiplex PCR using primer sets for Campylobacter-specific 16S rRNA gene and four Campylobacter species-specific primers were used (Table 1).

### Statistical Analysis

Statistical analysis was performed using chi-square test or student's t-test with GraphPad Prism (Version 5.01, GraphPad Software, Inc., Sand Diego, Calif.).

TABLE 1
Primers used in this study

| | | | | SEQ | |
|---|---|---|---|---|---|
| Genus | Gene | Primer | Sequence (5′-3′) | ID NO | Reference |
| Escherichia | uidA | uidA- | AGGTGGTTGCAACTGG | 1 | This study |
| coli | | qPCR-F | ACAA | | |
| | | uidA- | TCAGGAACTGTTCGCC | 2 | |
| | | qPCR-R | CTTC | | |
| Campylobacter | 16s rRNA | 16s- | ATAAGCACCGGCTAAC | 3 | This study |
| spp. | | qPCR-F | TCCG | | |
| | | 16s- | TTCCATCTGCCTCTCC | 4 | |
| | | qPCR-R | CTCA | | |
| | 16s rRNA | C412F | GGATGACACTTTTCGG | 5 | (Linton et al. Res |
| | | | AGC | | Microbiol 147, 707-718 |
| | | C1228R | CATTGTAGCACGTGTG | 6 | (1996)) |
| | | | TC | | |
| | cj0414 | C-1 | CAAATAAAGTTAGAGG | 7 | (Wang et al. Journal |
| | | | TAGAATGT | | of Rapid Methods and |
| | | C-3 | CCATAAGCACTAGCTA | 8 | Automation in |
| | | | GCTGAT | | Microbiology 1, 101- |
| | | | | | 108 (1992)) |
| | hipO | hipO-F | GCAAAATCCACAGCTT | 9 | (Kim et al. |
| | | | CATCGT | | Microorganisms 7, 433 |
| | | hipO-R | GGAAGGGGTGGTCATG | 10 | (2019)) |
| | | | GAAG | | |
| | ask | CC18F | GGTATGATTTCTACAA | 11 | (Linton et al. Res |
| | | | AGCGAG | | Microbiol 147, 707-718 |
| | | CC519R | ATAAAAGACTATCGTC | 12 | (1996)) |
| | | | GCGTG | | |

TABLE 2
Characterization of phages

| | | Size (nm; n = 3) | | Genome features | | | | |
|---|---|---|---|---|---|---|---|---|
| Phage | Isolation source | Head | Tail | Size (bp) | GC (%) | ORF^{a} | tRNA | Accession No. |
| JEP1 | Retail chicken | 79.6 ± 1.9 | 101.0 ± 3.8 | 143,610 | 43.54 | 223 | 5 | MT740314 |
| JEP4 | Chicken feces | 106.3 ± 5.5 | 102.9 ± 2.6 | 39,195 | 47.05 | 61 | 0 | MT740315 |
| JEP6 | Pig feces | 109.1 ± 1.7 | 110.3 ± 2.3 | 170,340 | 35.31 | 274 | 7 | MT764206 |
| JEP7 | Retail duck | 103.9 ± 5.2 | 95.2 ± 2.4 | 52,936 | 45.94 | 71 | 0 | MT764207 |
| JEP8 | Retail chicken | 96.1 ± 2.5 | 95.9 ± 3.5 | 165,295 | 40.47 | 272 | 0 | MT764208 |

^{a}open reading frame

TABLE 3A

| Phylogenetic | | | Phage | | | | |
|---|---|---|---|---|---|---|---|
| group | New stock no. | ESBL type | JEP1 | JEP4 | JEP6 | JEP7 | JEP8 |
| A | 1 | CTX-M-65 | \| | ++ | — | — | — |
| | 2 | CTX-M-65 | \| | ++ | — | — | — |
| | 3 | CTX-M-65 | \| | ++ | ++ | — | +++ |
| | 4 | CTX-M-55 | — | — | — | — | ++ |
| | 5 | CTX-M-65 | — | +++ | — | — | — |
| | 6 | CTX-M-55, CTX-M-65 | \| | + | \| | ++ | — |
| | 7 | CTX-M-65 | \| | + | + | + | — |
| | 8 | CTX-M-55, TEM-1 | — | — | — | — | — |
| | 9 | CTX-M-55, TEM-116 | ++ | — | \| | ++ | +++ |
| | 10 | CTX-M-65 | \| | ++ | ++ | — | +++ |
| | 11 | CTX-M-65 | \| | +++ | ++ | — | ++ |
| | 12 | CTX-M-65 | + | ++ | \| | ++ | — |
| | 13 | CTX-M-65 | — | +++ | — | — | — |
| | 14 | CTX-M-65 | \| | +++ | — | — | — |
| | 15 | CTX-M-65 | \| | +++ | — | — | — |
| | 16 | CTX-M-65, TEM-116 | — | + | \| | \| | — |
| | 17 | CTX-M-55, TEM-1 | — | +++ | +++ | — | \| |
| | 18 | CTX-M-15, TEM-1 | — | +++ | +++ | — | \| |
| | 19 | CTX-M-65 | — | +++ | — | — | — |
| | 20 | CTX-M-55, CTX-M-65 | \| | ++ | ++ | — | +++ |
| | 21 | CTX-M-55, TEM-1 | — | — | \| | ++ | — |
| | 22 | CTX-M-65, TEM-116 | \| | + | \| | ++ | — |
| | 23 | CTX-M-65, TEM-116 | ++ | ++ | \| | ++ | — |
| | 24 | CTX-M-14 | — | +++ | ++ | \| | \| |
| | 25 | CTX-M-14 | — | +++ | \| | — | — |
| | 26 | CTX-M-55, TEM-1 | \| | — | \| | \| | — |
| | 27 | CTX-M-14, TEM-1 | — | ++ | — | — | — |
| | 28 | CTX-M-65, TEM-1 | — | — | +++ | \| | — |
| | 29 | CTX-M-65, TEM-116 | \| | +++ | ++ | — | \| |
| | 30 | CTX-M-65, TEM-116 | — | +++ | — | — | — |
| | 31 | CTX-M-65, TEM-116 | \| | +++ | — | — | — |
| | 32 | CTX-M-55, TEM-1 | — | — | — | — | — |
| | 33 | CTX-M-65, TEM-116 | — | — | \| | \| | — |
| | 34 | CTX-M-55, TEM-116 | \| | +++ | +++ | \| | — |
| | 35 | CTX-M-65 | — | +++ | — | — | — |
| | 36 | CTX-M-65 | \| | +++ | — | — | — |
| | 37 | CTX-M-14 | — | — | — | ++ | — |
| | 38 | CTX-M-55, TEM-116 | — | — | \| | — | ++ |
| B1 | 39 | CTX-M-65, TEM-1 | +++ | — | — | ++ | — |
| | 40 | CTX-M-14, TEM-116 | +++ | ++ | +++ | +++ | — |
| | 41 | CTX-M-14 | +++ | \| | +++ | ++ | — |
| | 42 | CTX-M-14, TEM-1 | \| | \| | +++ | — | \| |
| | 43 | CTX-M-65, OXA-1, TEM-1 | +++ | — | — | ++ | — |
| | 44 | CTX-M-65, OXA-1, TEM-1 | +++ | — | — | ++ | — |
| | 45 | CTX-M-65, OXA-1, TEM-1 | +++ | — | — | ++ | — |
| | 46 | CTX-M-65, OXA-1, TEM-1 | ++ | — | — | + | — |
| | 47 | CTX-M-55, CTX-M-65, | +++ | — | — | ++ | — |
| | | OXA-1, TEM-1 | | | | | |
| | 48 | CTX-M-65, OXA-1, TEM-1 | +++ | — | — | ++ | + |
| | 49 | CTX-M-14, TEM-1 | \| | — | — | \| | + |
| | 50 | CTX-M-27 | — | — | — | — | — |
| | 51 | CTX-M-14 | — | — | — | + | — |
| B2 | 52 | CTX-M-55, TEM-116 | — | — | +++ | — | — |
| | 53 | CTX-M-15, TEM-135 | \| | — | — | — | — |
| | 54 | CTX-M-55, TEM-116 | — | — | +++ | \| | — |
| | 55 | CTX-M-14, TEM-1 | — | — | +++ | \| | — |
| D | 56 | CTX-M-14, TEM-1 | +++ | +++ | — | ++ | — |
| | 57 | CTX-M-14, TEM-1 | +++ | +++ | — | ++ | — |
| | 58 | CTX-M-55, TEM-116 | \| | — | ++ | — | — |
| | 59 | CTX-M-55 | ++ | +++ | ++ | ++ | — |
| E | 60 | CTX-M-65 | — | — | +++ | \| | — |
| | 61 | CTX-M-65 | \| | — | ++ | +++ | — |
| | 62 | CTX-M-65 | — | — | ++ | +++ | — |
| | 63 | CTX-M-14 | — | — | — | — | +++ |
| | 64 | CTX-M-14 | — | — | — | — | +++ |
| | 65 | CTX-M-14, TEM-116 | — | — | — | — | +++ |
| F | 66 | CTX-M-55, TEM-1 | — | — | — | \| | +++ |
| Unknown | 67 | CTX-M-65, TEM-1 | \| | \| | ++ | — | — |

TABLE 3B

| Phylogenetic group | JEP1 | JEP4 | JEP6 | JEP7 | JEP8 |
|---|---|---|---|---|---|
| A | 3/38 (7.9%) | 28/38 (73.7%) | 11/38 (28.9%) | 8/38 (21.1%) | 7/38 (18.4%) |
| B1 | 9/13 (69.3%) | 1/13 (7.7%) | 3/13 (23.1%) | 10/13 (76.9%) | 2/13 (15.4%) |
| B2 | 0/4 (0.0%) | 0/4 (0.0%) | 3/4 (75.0%) | 0/4 (0.0%) | 0/4 (0.0%) |
| D | 3/4 (75.0%) | 3/4 (75.0%) | 2/4 (50.0%) | 3/4 (75.0%) | 0/4 (0.0%) |
| E | 0/6 (0.0%) | 0/6 (0.0%) | 3/6 (50.0%) | 2/6 (33.3%) | 3/6 (50.0%) |
| F | 0/1 (0.0%) | 0/1 (0.0%) | 0/1 (0.0%) | 0/1 (0.0%) | 1/1 (100.0%) |
| Unknown | 0/1 (0.0%) | 0/1 (0.0%) | 1/1 (100.0%) | 0/1 (0.0%) | 0/1 (0.0%) |
| | 15/67 (22.4%) | 32/67 (47.8%) | 23/67 (34.3%) | 23/67 (34.3%) | 13/67 (19.4%) |

Different Efficiencies in Phage Infection of ESBL-Producing E. coli Depending on the Bacterial Phylogenetic Group.

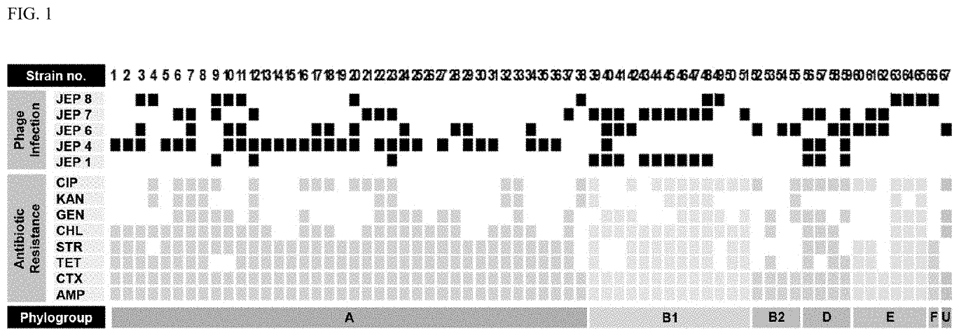

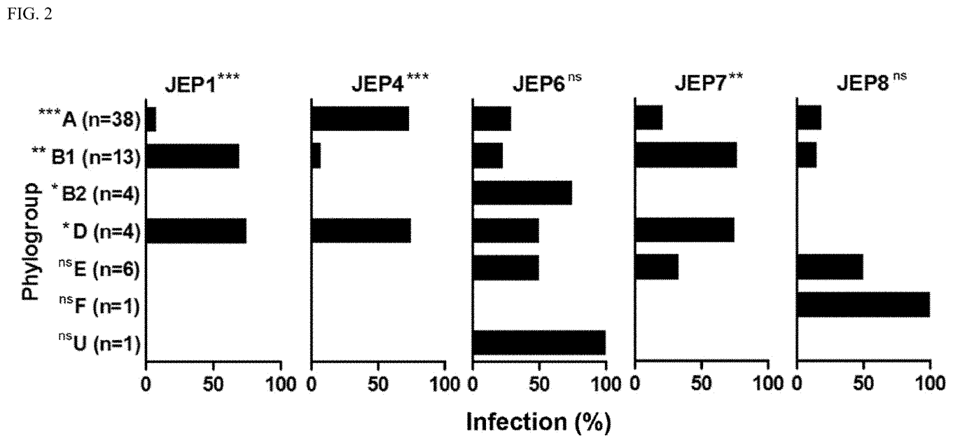

The construction of a phage cocktail was initiated by evaluating the infectivity of eight E. coli phages, which were isolated from meat and animal fecal samples, on 67 ESBL-producing E. coli isolates from retail chicken (Park et al. J Glob Antimicrob Resist 17, 216-220 (2019)). In addition to the harboring of ESBL genes, the E. coli strains were also resistant to antibiotics of other classes, such as chloramphenicol, tetracycline, and aminoglycosides (FIG. 1). The extensive phage infection assay led to the identification of five phages that infected ESBL-producing E. coli at high infection frequencies: JEP1, JEP4, JEP6, JEP7, and JEP8 (FIG. 1 and FIGS. 11-15 (phage maps)). The five phages exhibited differential infectivity depending on the phylogenetic group of E. coli. For instance, JEP4 phage primarily infects ESBL-producing E. coli strains belonging to phylogroup A (74%, 28/38), whereas JEP1 and JEP7 phages demonstrated high infection preference to group B1 strains (69% ( 9/13) and 77% ( 10/13), respectively) and D (75%, ¾) but showed little inhibitory effect on other phylogenetic groups (FIG. 1, FIG. 2, and Table 3). JEP1, JEP4, JEP6, and JEP7 infected a couple of the major phylogenetic groups (A, B1, B2, and D) (Park et al. J Glob Antimicrob Resist 17, 216-220 (2019), Kim et al. Microorganisms 7, 344 (2019)), and JEP8 was the sole phage able to infect E. coli in phylogroup F (100%, 1/1), even though there was only a single strain in the minor group (FIG. 1 and FIG. 2). A cocktail of the five phages allowed for the infection of 91% ( 61/67) of ESBL-producing E. coli strains (FIG. 1).

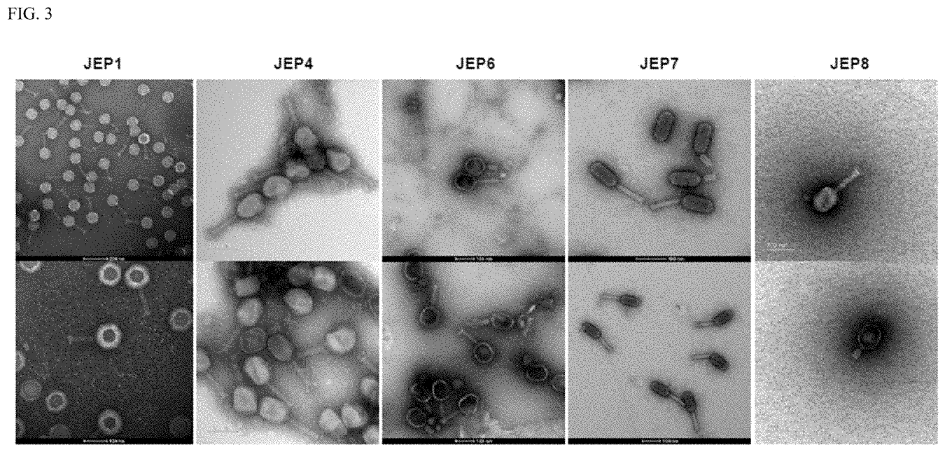

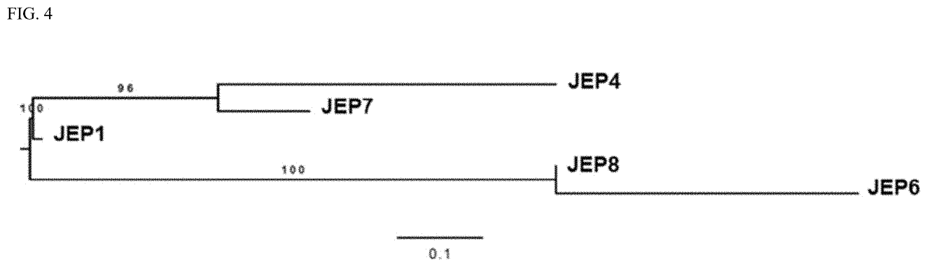

A morphological analysis using transmission electron microscopy revealed that the phages had a big head and an inflexible/contractile tail and belonged to the Myoviridae family (FIG. 3, Table 2). Also, whole-genome sequencing analysis showed that the phages have various genome sizes ranging from 39 kb (JEP4) to 170 kb (JEP6) (Table 2). The phylogenetic analysis of the five phages using the Genome BLAST Distance Phylogeny (GBDP) approach (Meier-Kolthoff et al. BMC bioinformatics 14, 60 (2013)) generated four clusters at the genus level, suggesting that the phages are relatively phylogenetically distant (FIG. 4).

Antimicrobial Synergy Between Phages and Antibiotics Against ESBL-Producing E. coli

Since Bolton Campylobacter-selective broth (BB) and Preston Campylobacter-selective broth (PB) are recommended by the international Organization for Standardization (ISO) for selective isolation of Campylobacter (10272-1, (2017)), the efficiency of the phage cocktail in the inhibition of ESBL-producing E. coli was determined using BB and PB. One strain was randomly selected from each of the major phylogenetic groups (A, B1, B2, and D), and the experiment was conducted with a mixture of the four strains (E. coli E20, 41, 55, and 59). The mixed-culture of ESBL-producing E. coli strains was enriched in BB or PB with different multiplicity of infection (MOI) values (10⁰ to 10⁶) of the phage cocktail. No significant colony forming units (CFU) reduction was observed in BB at low MOI values (<10²), and the phage cocktail reduced the level of ESBL-producing E. coli strains only marginally (0.73 and 1.07 log CFU/ml) even at high MOIs (10⁴ and 10⁶) (FIG. 5A). However, the phage cocktail reduced the ESBL-producing E. coli strains in PB in proportion to the increase in MOI, and ESBL-producing E. coli strains were undetectable (<10 CFU/ml) at the MOI of 10⁶ (FIG. 5B). The efficacy of E. coli inhibition by the phage cocktail varied depending on the type of selective media.

Because the selective pressure of the enrichment media is determined by antibiotic supplements in the media, the inhibition efficacy of the phage cocktail was measured in the presence of four antibiotics (cefoperazone, vancomycin, polymyxin B, and rifampicin) used in BB and PB. When the antibiotics were used without the phage cocktail, the MICs of cefoperazone, vancomycin, polymyxin B, and rifampicin were >512 μg/mL, 64 μg/mL, 4 μg/mL, and 32 μg/mL, respectively (FIG. 5C). A checkerboard assay was performed to examine the antimicrobial activity of the phage cocktail in combination with antibiotics. Interestingly, the phage cocktail inhibited ESBL-producing E. coli differentially depending on the antibiotic. The most significant synergistic inhibition was observed when the phage cocktail was used with rifampicin, an antibiotic used in PB (FIG. 5C). The phage cocktail reduced the MIC of vancomycin by two-fold regardless of MOI and that of polymyxin B by four-fold at high MOI levels (FIG. 5C). Although the MIC of cefoperazone was extremely high (>512 μg/ml) due to the production of ESBL in E. coli, the MIC was gradually reduced when the MOI was increased (FIG. 5C). These results demonstrated that phage cocktails can produce antimicrobial synergy in combination with antibiotics, though inhibition efficacy is affected by antibiotic paired with the phage cocktail. These results suggest the more effective reduction in the level of ESBL-producing E. coli strains in PB than BB may be due to synergy between phages and rifampicin, an antimicrobial supplement in PB.

Phage-Mediated Inhibition of ESBL-Producing E. coli During Selective Enrichment

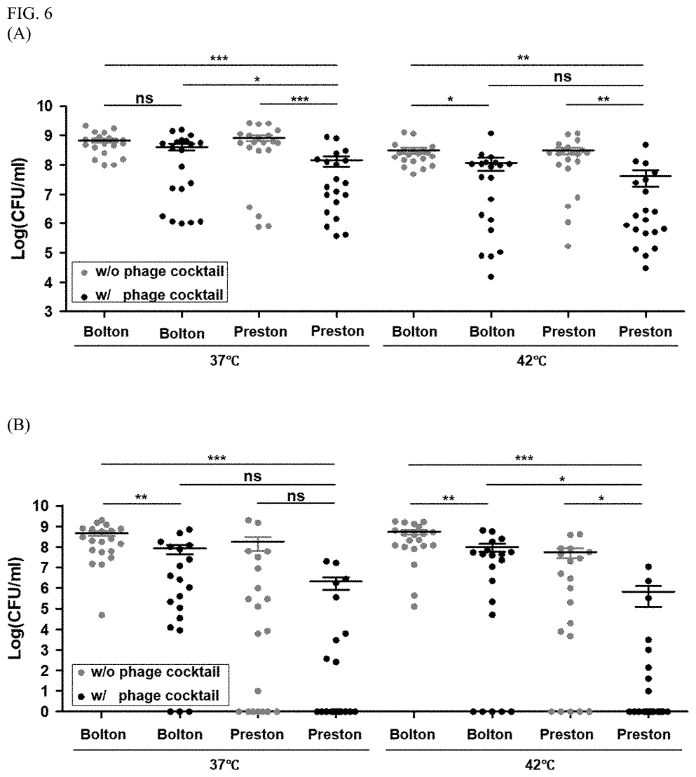

To evaluate the effect of the phage cocktail on selective enrichment of Campylobacter, isolation frequency under eight different enrichment conditions that combined two selective media (BB and PB) at different temperatures (37° C. and 42° C.) with or without the phage cocktail were measured. One hundred sixty samples from 20 chicken samples prepared under the eight enrichment conditions were analyzed for the level of E. coli using qPCR and the viable count of E. coli resistant to the third-generation cephalosporin. The results of qPCR analysis showed that all tested samples were positive for E. coli. Whereas the phage cocktail reduced the level of E. coli in BB at 42° C., E. coli was significantly inhibited by the cocktail in PB both at 37° C. and 42° C. (FIG. 6A). Consistently, the supplementation with the phage cocktail significantly reduced the level of ESBL-producing E. coli strains in PB compared to BB (FIG. 6B). Even though the phage cocktail reduced the level of ESBL-producing E. coli strains at 42° C. in both BB and PB, the CFU reduction was more significant in PB than BB (FIG. 6B). The level of Campylobacter in the enrichment samples was also measured. The highest level of Campylobacter was observed when the samples were enriched in PB at 42° C. (FIG. 7). However, the supplementation with the phage cocktail did not increase the level of Campylobacter with a statistical significance. These results suggest that the efficacy of the phage cocktail was influenced by treatment temperatures and selective media.

Optimization of Culture Conditions for Campylobacter Isolation Using E. coli Phages

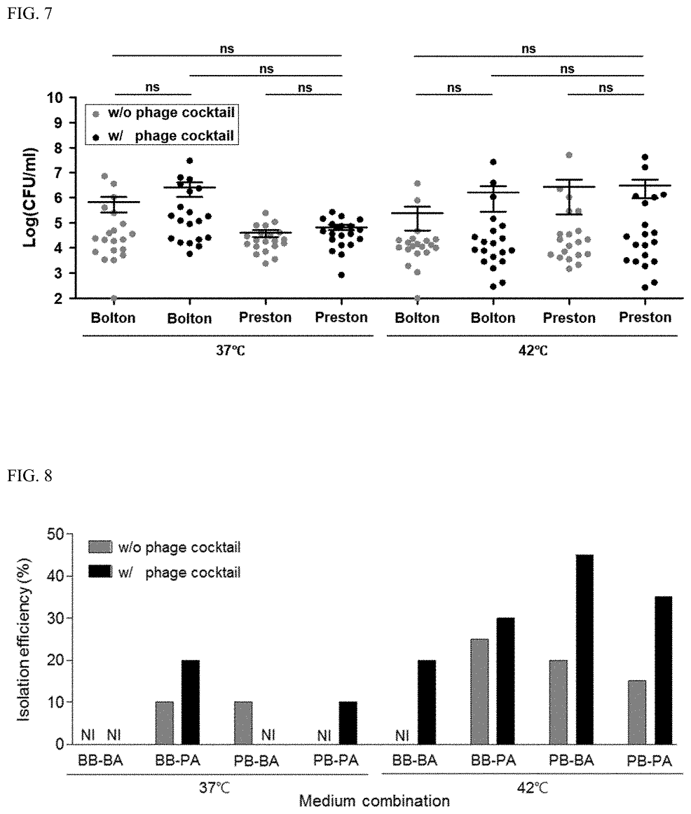

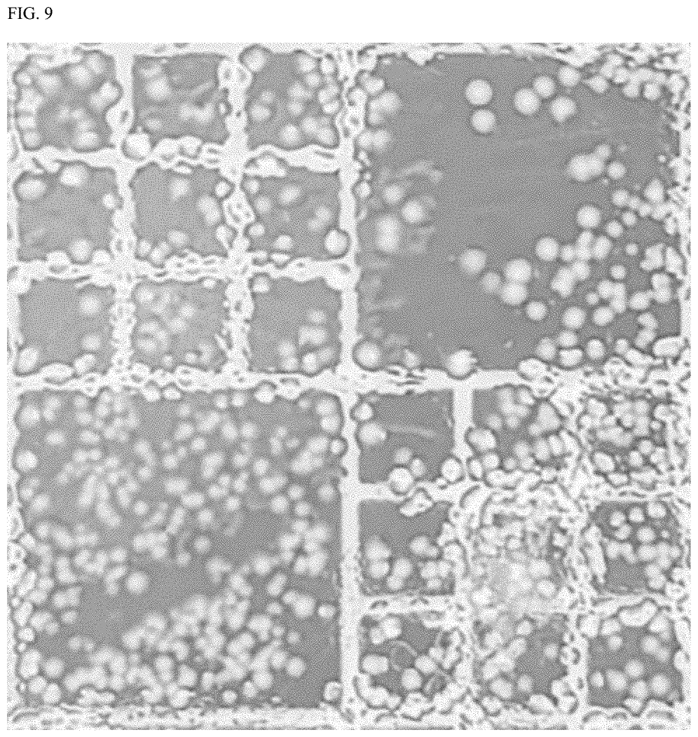

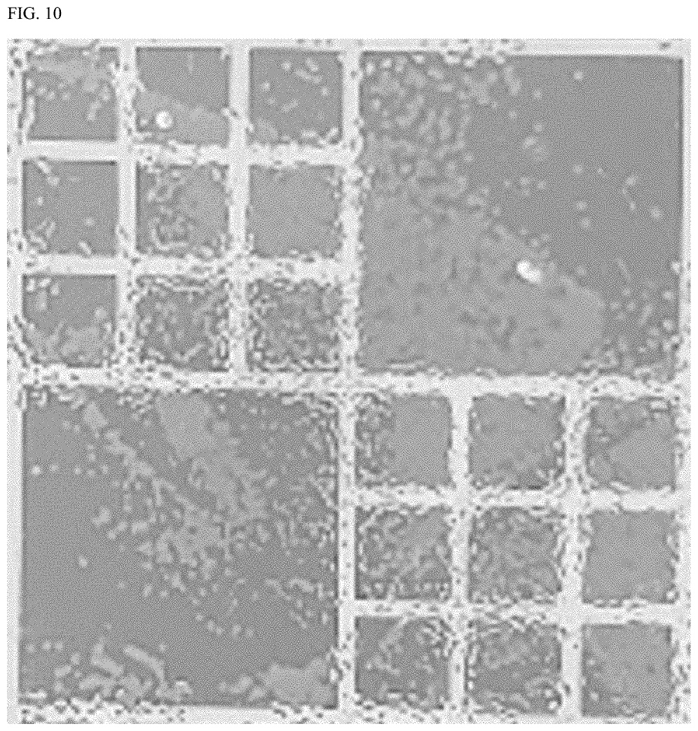

To assess how the phage cocktail promotes Campylobacter isolation, the isolation frequencies of 16 different isolation conditions were also measured, including the first enrichment media (BB and PB), the first selective solid media (Bolton agar (BA) and Preston agar (PA)), temperatures (37° C. and 42° C.), and with or without the phage cocktail. Each combination exhibited different levels of isolation frequency with a wide range of variation from 0% to 80%, and Campylobacter was not isolated from the samples of five combinations (FIG. 8). Although the supplementation of the phage cocktail significantly increased the isolation frequency at 42° C. in the four combinations of selective media, the highest frequency was observed when the samples were enriched at 42° C. in PB supplemented with the phage cocktail and subsequent culture on BA (FIG. 7). This can be ascribed to the significant inhibition of ESBL-producing E. coli strains by the phage cocktail in PB at 42° C. (FIG. 6). In addition to the increased isolation frequency, the phage cocktail made it convenient to identify Campylobacter colonies easily. Without the phage treatment, agar plates were heavily covered with E. coli colonies (FIG. 9A), whereas with phage cocktail treatment, Campylobacter colonies were distinguishable (FIG. 9B).

Clermont phylotyping classifies E. coli into four major (A, B1, B2 and D) and two minor groups (E and F). Phylogenetic groups of E. coli are related to certain pathotypes, such as extraintestinal pathogenic E. coli (ExPEC). Whereas phylogroups A and B1 are predominant among commensal E. coli strains, ExPEC strains predominate phylogroups B2 and D. Also, E. coli phylotyping exhibits bacterial association with specific hosts. Phylogroups A and B2 predominate in human strains, while phylogroup B1 strains are predominant in animal strains, followed by phylogroup A strains. E. coli isolates from chickens mostly belong to the phylogroups A and B1. ESBL-producing E. coli strains isolated from retail chicken dominantly belong to phylogroups A and B1, followed by phylogroups B2, D, E, and F.

The infection efficacy of phages varied depending on the phylogenetic group of ESBL-producing E. coli (FIG. 1 and FIG. 2). JEP1 and JPE7 showed high infection efficiency for E. coli strains belonging to phylogroups B1 and D, and JEP4 infected 74% and 75% of E. coli strains in phylogroups A and D, respectively (FIG. 1 and FIG. 2). JEP6 and JEP8 were able to infect hosts belonging to phylogroups B2 and F at high efficiencies (FIG. 1 and FIG. 2). To develop a phage cocktail capable of infecting poultry isolates of E. coli with various genetic backgrounds, phages were used that can effectively infect the major phylogenetic groups of E. coli strains isolated from poultry and increased phage efficiency to 91% (FIG. 1). Phage cocktails are typically constructed by testing host ranges with relatively limited numbers of bacteria, normally a few type strains. The screening of phage infectivity on an increased number of E. coli strains isolated from the same source—i.e., chicken carcasses in this study—revealed that E. coli phages have different infection efficiency depending on the phylogenetic group.

Bacteria rapidly develop phage resistance by various mechanisms. To control bacteria resistant to phage infection, phage cocktails are generally designed based on the use of different host receptors, the range of host bacteria, or the molecular mechanism of conversion to phage-resistant cells. In this study, a phage cocktail was constructed based on phylogenetic group-dependent infectivity even without identifying the host receptors of the phages, ascertaining the feasibility of the phylogenetic group-based design of phage cocktails. Given the association between E. coli phylogenetic group and hosts, depending on the application, one can construct phage cocktails based on phylogenetic group-dependent infection frequency. To develop phage cocktails to control human clinical strains, for instance, one can use phages effectively infecting E. coli in phylogroups A and B2, which are predominant in human strains. Also, one can consider making phage cocktails mainly targeting phylogroups B2 and D to control ExPEC strains.

Without wishing to be bound by any particular theory, the phylogenetic group-dependent phage efficiency may be due to the prevalence of phage receptors in phylogenetic groups. Because phage infection is initiated by phage adsorption to host receptors, mechanisms reduce, inhibit, or prevent phage infection include alteration of phage receptors (e.g., through spontaneous mutation) on the surface of host bacteria, the removal of receptor genes by an insertion sequence, and the interruption of phage access to host receptors. Phage receptors in E. coli are bacterial surface structures, such as the ferrichrome outer membrane transporter FhuA, the major outer membrane protein OmpC, surface glycoconjugates, and the 0 antigen of lipopolysaccharide (LPS). The prevalence of some of phage receptors is related to the phylogenetic group of E. coli. The distribution of genes encoding OMPs, fimbriae, or capsular proteins was significantly different for each phylogenetic group, and the phylogenetic group of E. coli is related to the type of the core oligosaccharide of LPS. Because phages use cell surface moieties as receptors, the host range of phages can alter if the prevalence of the receptors are varied in different phylogenetic groups.

The specificity of phage infection may provide advantages over conventional antimicrobial agents because phage therapy may eliminate the target bacteria without interfering with normal microflora. The infection specificity of phages, however, also creates limitations in inhibiting genetically diverse bacteria harboring various host receptors. In fact, the host range of phages is too narrow to cope with bacteria from food, environmental niches, and infection sites. Phages can be genetically engineered to modify host range by mutating receptor binding domains. The results in this study suggest that the selection of phages based on the bacterial phylogenetic groups can be a novel strategy for the construction of broad host range phage cocktails while maintaining the infection specificity towards target bacteria without genetic modifications.

In the selective enrichment as a model system, phage therapy was combined complementarily with antibiotics to inhibit antibiotic-resistant bacteria (FIG. 5C). The antimicrobial synergy between phages and antibiotics was affected by the antibiotic paired with phages due, in part, to antibiotic resistance in target bacteria. ESBL-producing E. coli is resistant to cefoperazone, a third-generation cephalosporin drug, and vancomycin is not effective against Gram-negative bacteria. When combined with vancomycin, thus, phages generated antimicrobial synergy only marginally, two-fold reduction in the MIC regardless of MOI, and the MIC of cefoperazone was reduced when phages were treated at high MOIs (FIG. 5C). The most significant antimicrobial synergy was observed when the phages were coupled with rifampicin, which inhibit RNA synthesis. The phage cocktail reduced the MIC of rifampicin by 32-fold at an MOI of 10 (FIG. 5C). Because rifampicin is one of the antimicrobials in PB, the phage cocktail reduced the level of ESBL-producing E. coli more significantly in PB than BB (FIG. 6B). The phage cocktail decreased only four-fold reduction in the MIC of polymyxin B at high MOIs (100 and 1000), although polymyxin B is an antimicrobial peptide active against Gram-negative bacteria, suggesting the mode of antimicrobial action may also contribute to antimicrobial synergy with phages.

The isolation and cultivation of fastidious bacteria pose a challenge to microbiology laboratories because providing nutrients cannot ensure the growth of fastidious target bacteria. In addition, fastidious bacteria are often outgrown by competitive microbiota during isolation. Thus, suppressing competitors can contribute to the promotion of the growth of fastidious bacteria. For this purpose, culture media are supplemented with antibiotics to allow for the selective growth of target bacteria. However, if some of indigenous bacteria in background microbiota are resistant to antimicrobial supplements used in selective media, the resistant bacteria compete with and interfere with the growth of the target bacteria. Due to the rise in antibiotic resistance, the efficacy of antibiotics used in selective enrichment will become more and more limited. This study demonstrates that these limitations may be overcome by using phages as antimicrobial agents complementarily to antimicrobial supplements.

Multiple environmental factors are involved in the efficacy of Campylobacter isolation. Antibiotics with unique mode of action used in different selective media substantially affected the level of competitive bacteria (FIG. 5C). The sequence of pairing selective media is another important factor determining the isolation frequency. Selective enrichment with BB with the phage cocktail followed by plating on PA exhibited 40% of isolation efficiency at 37° C., whereas enrichment with PB with the phage cocktail followed by plating on BA showed the highest (80%) efficiency (FIG. 8). Additionally, culture temperature is also an important determinant affecting the isolation efficiency. Overall, the frequency of Campylobacter isolation was higher at 42° C. than 37° C. because the phage cocktail inhibited ESBL-producing E. coli more efficiently at 42° C. than 37° C. (FIG. 6). Some medium combinations (BB-PA, PB-BA, and PB-PA) showed high isolation frequencies even without the phage cocktail at 42° C. when compared to the samples prepared at 37° C., which may be because microbiota that were developed under different conditions may affect the efficiency of Campylobacter isolation. Since human infection with Campylobacter is mainly caused by the consumption of contaminated poultry, in many countries, baseline levels of food contamination and risk assessment are used establish food safety policies. The results in this study show that the frequency of Campylobacter isolation ranged from 0% to 80%, depending on the isolation conditions, even though the same samples and media were used (FIG. 8), suggesting standardized protocols need to be used to compare the levels of Campylobacter contamination in different countries.

### Example 2—Inhibition of Antibiotic-Resistant E. coli in Chicken Carcasses Using Broad Host Range Bacteriophage Cocktail

### Phage Cocktail

A phage cocktail including five Myoviridae phages (JEP1, JEP4, JEP6, JEP7, and JEP8) was produced as described in Example 1.

### Challenge Assay

After the cultivation of E. coli strains (E20, 41, 55, and 59), which were selected from the major phylogenetic groups (A, B1, B2, and D), to an optical density at 600 nm (OD₆₀₀) of 0.5, the mixed-culture of E. coli strains was diluted and added to 4 mL of LB broth at 10⁵ colony forming units (CFU)/mL followed by addition of SM buffer (control) or phage cocktail at 10⁸ plaque forming units (PFU)/mL (multiple of infection (MOI) 10³).

### Food Application

Chicken carcasses were purchased, and the skin was cut into a 2 cm×2 cm squares. For decontamination, the chicken skin samples were UV-treated on both sides for 30 minutes in a bio-safety cabinet. The mixed-culture of ESBL-producing E. coli strains was prepared by mentioned above and diluted to 8×10⁶ CFU/ml. Then 50 μL of mixed-culture of E. coli strains was spotted onto a 2 cm×2 cm chicken skin to achieve the final inoculum level of approximately 5 log CFU/cm² of the chicken sample. For the negative control group, the same volume of PBS was added to a chicken skin. Samples were dried in bio-safety cabinet for 30 minutes. Then, 100 μL of phage cocktails (MOI of 10³) or SM buffer as positive control were spotted onto each samples, and incubated at 4° C. and 25° C. At three hours, six hours, and 12 hours of incubation, each sample was mixed with 0.1% buffered peptone water (BPW) and homogenized by vortexing for two minutes in 50 mL tube. The homogenate was centrifuged at 10,000×g for five minutes, and pellets were resuspended with 10 mL of BPW. After 10-fold serial dilution, the culture was plated onto LB agars.

Inhibition of ESBL-Producing E. coli by the Phage Cocktail Against on Chicken Skin

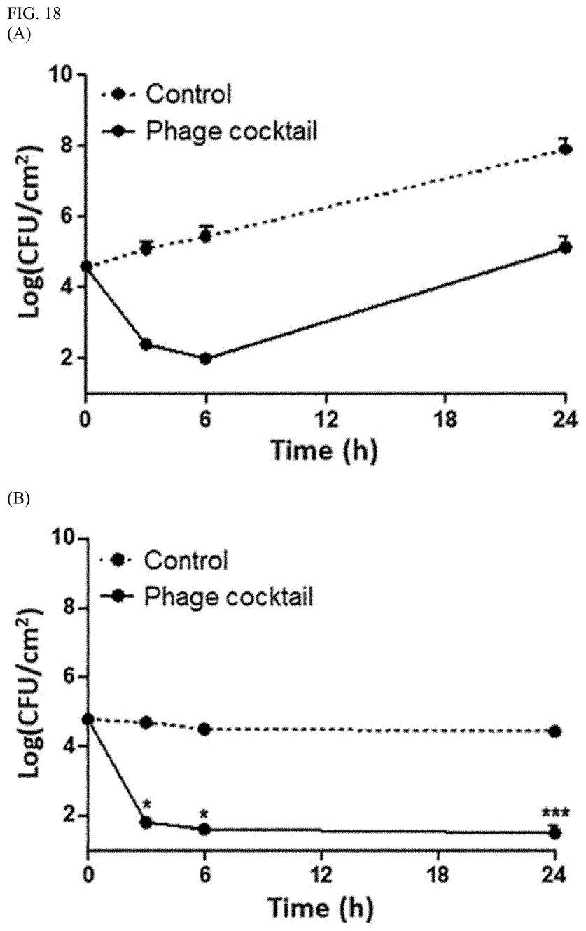

The inhibition efficiency of the phage cocktail was tested on raw chicken skin. To mimic the pathogen-contaminated condition, raw chicken skin samples were artificially spiked with the mixed-culture of ESBL-producing E. coli strains at 5 log CFU/cm² (FIG. 18). As shown in FIG. 18A, growth of the mixed-culture of ESBL-producing E. coli strains was observed in the control sample incubated at 25° C. without treatment with the phage cocktail (FIG. 18A). In contrast, when a raw chicken skin sample was treated with phage cocktail (MOI of 10³) and incubated at 25° C., a rapid reduction of the mixed-culture of ESBL-producing E. coli strains was observed at three hours and six hours of incubation (FIG. 18A). After six hours of incubation, the growth of the mixed-culture of ESBL-producing E. coli strains in sample increased (FIG. 18A). When incubated at 4° C. (FIG. 18B), the CFU of the mixed-culture of ESBL-producing E. coli strains in the control sample appeared unchanged during the incubation time, the treatment of phage cocktail significantly reduced the mixed-culture of ESBL-producing E. coli strains compared to the control (FIG. 18B). Treatment of phage cocktail gradually reduced the mixed-culture of ESBL-producing E. coli strains at three, six, and 24 hours (FIG. 18B).

These results indicate that the phage cocktail inhibited ESBL-producing E. coli more efficiently at 4° C. than 25° C. on chicken skin.

To inhibit ESBL-producing E. coli on chicken carcasses, an E. coli phage cocktail having phages with different infection efficiency depending on the phylogenetic group of E. coli was designed. As further described in Example 1, the phage cocktail includes phages that preferentially infect the major phylogroups (A, B1, B2, and D) and those infecting minor groups.

The phage cocktail significantly reduced antibiotic-resistant E. coli on chicken skin. The incubation temperature affected adsorption and efficacy of phage. In a previous study, the level of bacterial reduction with phage cocktail was more effective at 25° C. than at 5° C. on raw chicken meat (Hoang Minh et al. LWT—Food Science and Technology 71, 339-346 (2016)). In contrast, the cocktail of the five phages constructed in this study was more effect at 4° C. than at 25° C. (FIG. 18). Without wishing to be bound by any particular theory, the emergence of phage resistant bacteria can be accelerated at high temperatures by rapid cell growth and limited restriction-modification systems at low temperatures. These effects may mean the phage cocktail is more useful to industrial settings because raw chicken products are processed and distributed at refrigeration temperatures. For example, the phage cocktail may be applied to a food product—e.g., chicken meat—during food processing and storage. In addition, the phage cocktail may be used as a therapeutic to treat or inhibit infection with antibiotic-resistant, pathogenic E. coli strains in animals and humans.

### Example 3—Buffer Composition to Intensify the Efficacy of Phage Cocktail

Phage infection is initiated by phage binding to host receptors on the surface of target bacteria. Because phage recognition is mediated by receptor-ligand interaction in this Example whether the activity of the E. coli phage cocktail may be further improved by controlling the composition of divalent cationic ions was explored.

Phage infectivity was screened in the presence of different concentrations of CaCl₂), MgCl₂, CuCl₂, and MnCl₂, which are frequently used as cofactors in biochemical reactions. For the testing, a mixed culture of ESBL-producing E. coli (E20, E41, E55, E59), as described in Example 1, was used.

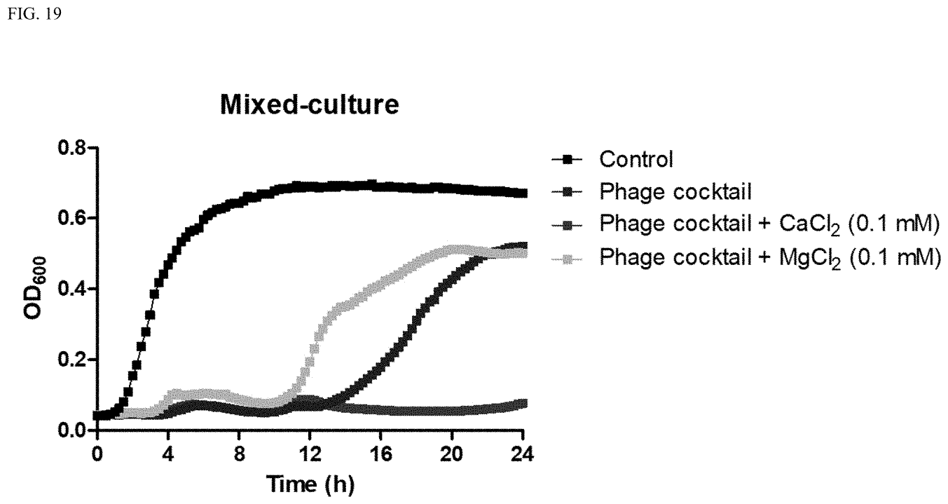

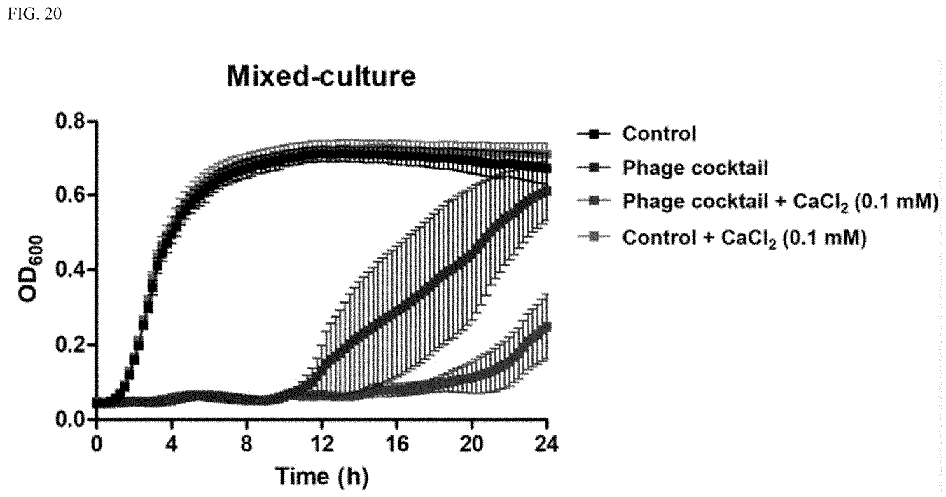

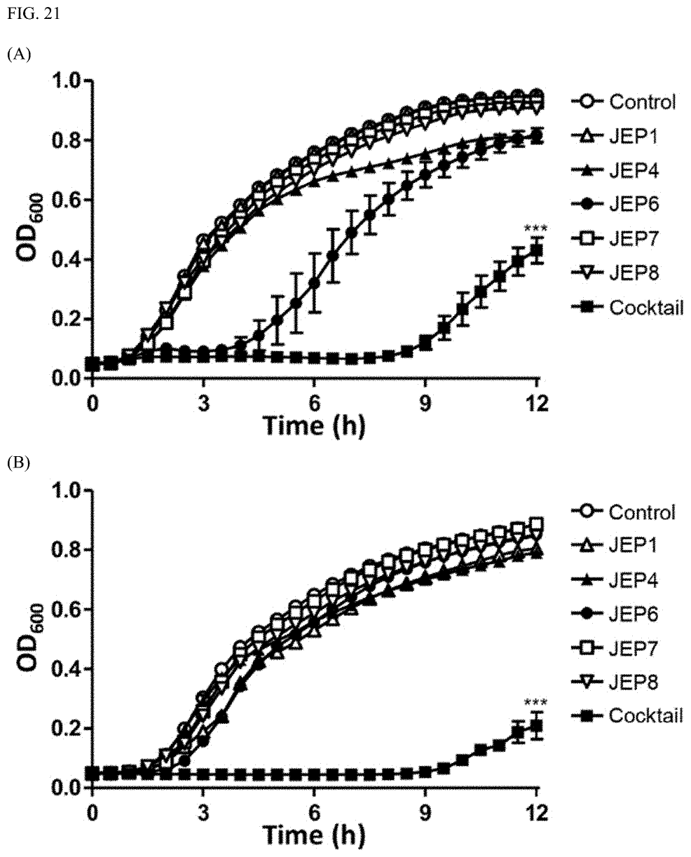

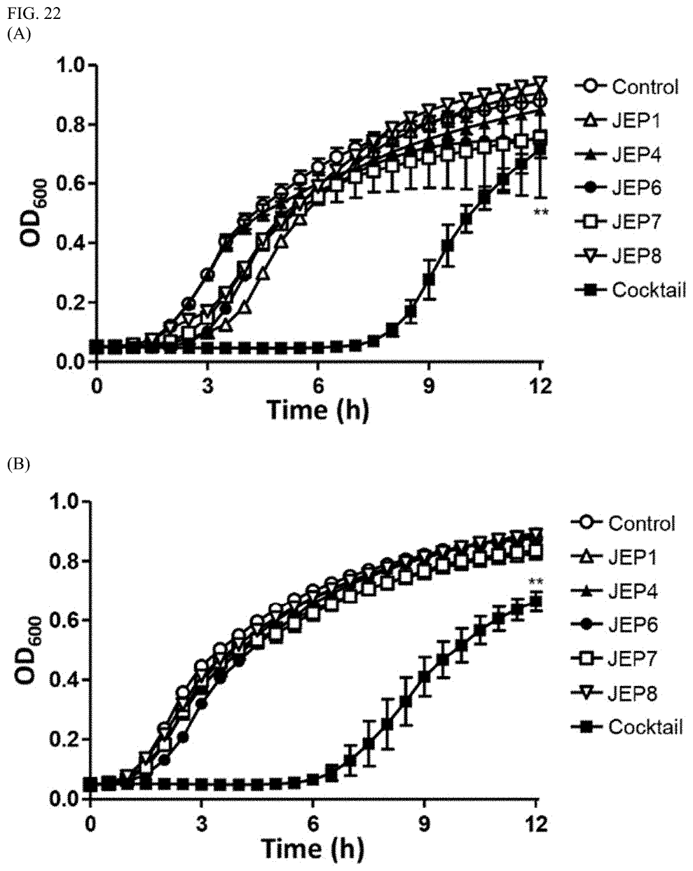

In the absence of a cationic ion, the phage cocktail effectively suppressed the growth of the mixed culture of ESBL-producing E. coli strains belonging to phylogenetic groups A, B1, B2, and D for 12 hours (FIG. 19B). Different cationic ions affected the ability of the phage cocktail to inhibit E. coli, with MgCl₂ and CaCl₂) being the most effective (FIG. 19A). In presence of 0.1 mM CaCl₂), the phage cocktail postponed the development of resistance until 20 hours and significantly intensified phage inhibition (FIG. 20). The effect of the phage cocktail, in the presence and absence of CaCl₂), against individual components of the mixed culture is shown in FIG. 21 and FIG. 22.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A composition comprising JEP1 (deposited in the NCBI database under GENBANK accession number MT740314), JEP4 (deposited in the NCBI database under GENBANK accession number MT740315), JEP6 (deposited in the NCBI database under GENBANK accession number MT764206), JEP7 (deposited in the NCBI database under GENBANK accession number MT764207), or JEP8 (deposited in the NCBI database under GENBANK accession number MT764208), or a combination thereof.

2. The composition of claim 1, wherein the composition comprises two, three, four, or all five of the phages.

3. The composition of claim 1, wherein the composition comprises a divalent cationic ion.

4. The composition of claim 3, wherein the divalent cationic ion comprises Ca²⁺, Mg²⁺, Cu²⁺, or Mn²⁺, or a combination thereof.

5. The composition of claim 3, wherein the divalent cationic ion comprises Ca²⁺.

6. The composition of claim 1, wherein the composition comprises CaCl₂) in a range of 0.1 mM to 15 mM or in a range of 0.1 mM to 10 mM.

7. The composition of claim 1, wherein the composition comprises a Campylobacter-selective enrichment media.

8. A method comprising adding the composition of claim 1 to a Campylobacter-selective enrichment media.

9. The method of claim 8, wherein the method comprises
adding a sample to the Campylobacter-selective enrichment media, and
incubating the Campylobacter-selective enrichment media at a temperature of at least 30° C., at least 33° C., at least 35° C., at least 37° C., at least 40° C., or at least 42° C., and up to 33° C., up to 35° C., up to 37° C., up to 40° C., up to 42° C., up to 45° C., or up to 47° C.; and/or
wherein the method comprises incubating the Campylobacter-selective enrichment media at a temperature of up to 33° C., up to 35° C., up to 37° C., up to 40° C., up to 42° C., up to 45° C., or up to 47° C.

10. The method of claim 8, wherein the method comprises adding an antibiotic to the Campylobacter-selective enrichment media.

11. The method of claim 9, wherein the sample comprises an isolate from a food source.

12. A method comprising adding JEP1 (deposited in the NCBI database under GENBANK accession number MT740314), JEP4 (deposited in the NCBI database under GENBANK accession number MT740315), JEP6 (deposited in the NCBI database under GENBANK accession number MT764206), JEP7 (deposited in the NCBI database under GENBANK accession number MT764207), or JEP8 (deposited in the NCBI database under GENBANK accession number MT764208), or a combination thereof to a composition.

13. The method of claim 12, wherein the method comprises adding two, three, four, or all five of JEP1, JEP4, JEP6, JEP7, and JEP8 to the composition.

14. The method of claim 12, wherein the composition comprises a food source.

15. The method of claim 14, wherein the food source comprises raw meat, an aquatic product, a raw vegetable, a retail-level ready-to-eat food, a frozen food, and/or a mushroom.

16. The method of claim 12, wherein the method comprises, after adding JEP1, JEP4, JEP6, JEP7, or JEP8, or a combination thereof to the composition, storing the composition at a temperature of up to 4° C., up to 7° C., up to 10° C., or up to 42° C.

17. A method comprising administering the composition of claim 1 to a subject.

18. The method of claim 17, wherein the composition is administered in an amount effect to control the growth of extended-spectrum beta lactamase (ESBL)-producing E. coli.

19. The method of claim 17, wherein the subject is a human, a companion animal, or a domesticated animal.

20. The method of claim 17, wherein the composition is administered to a subject suffering from an ESBL-producing E. coli infection.
